(19) United States
(12) Patent Application Publication
(10) Pub. No.: US 2023/0241328 A1
(43) Pub. Date: Aug. 3, 2023

(54) PRE-FILLED MULTI-FLUID MEDICAL DELIVERY ASSEMBLIES

(71) Applicant: Koska Family Limited, East Sussex (GB)

(72) Inventors: Marc Koska, Eastbourne (GB); Max Hannon, Flemingate (GB)

(21) Appl. No.: 18/119,114

(22) Filed: Mar. 8, 2023

Related U.S. Application Data
(63) Continuation of application No. PCT/IB2021/058168, filed on Sep. 8, 2021.
(60) Provisional application No. 63/075,790, filed on Sep. 8, 2020.

(51) Int. Cl. A61M 5/34; A61M 5/32

(52) U.S. Cl. CPC A61M 5/34; A61M 5/3245; A61M 2005/3252

(57) ABSTRACT

A pre-filled medical delivery system can have a blow-fill-seal (BFS) module and a mixing assembly. The BFS module can have first and second chambers, first and second sealed ports, and first and second actuation members. Each chamber can have a respective liquid agent therein. Each sealed port and each actuation member can be in fluid communication with a respective one of the chambers. The mixing assembly can be constructed for coupling to the BFS module. When coupled to the BFS module, the mixing assembly can breach the seals of the first and second ports and provide fluid communication therebetween. The disclosed systems, when assembled, can combine the liquid agents from the first and second chambers of the BFS component and deliver the combination as a single dose of a therapeutic agent (e.g., vaccine, drug, medicament, etc.) to a patient.

### CLAIM OF PRIORITY AND CROSS-REFERENCE TO RELATED APPLICATIONS

This application is a Continuation Application of PCT Application No. PCT/IB2021/058168, titled PRE-FILLED MULTI-FLUID MEDICAL DELIVERY ASSEMBLIES and filed on Sep. 8, 2021 in the name of Koska et al.; which PCT Application claims benefit of and priority under 35 U.S.C. § 119(e) to, and is a Non-provisional of U.S. Provisional Patent Application No. 63/075,790 filed on Sep. 8, 2020 and titled “PRE-FILLED DUAL-LIQUID MEDICAL DELIVERY ASSEMBLIES.” Each of these Applications is hereby incorporated by reference herein in its entirety and for all purposes.

### STATEMENT REGARDING FEDERALLY SPONSORED RESEARCH

This invention was made with government support under Award No. 75A50120C00007, awarded by the Department of Health and Human Services (HHS). The government has certain rights in the invention.

### BACKGROUND

Every year, millions of people become infected and die from a variety of diseases, some of which are vaccine-preventable. Although vaccination has led to a dramatic decline in the number of cases of several infectious diseases, some of these diseases remain quite common. In many instances, large populations of the world, particularly in developing countries, suffer from the spread of vaccine-preventable diseases due to ineffective immunization programs, either because of poor implementation, lack of affordable vaccines, or inadequate devices for administering vaccines, or combinations thereof.

Some implementations of immunization programs include administration of vaccines via a reusable syringe. However, in many situations, particularly in developing countries, the administration of vaccines occur outside of a hospital and may be provided by a non-professional, such that injections are given to patients without carefully controlling access to syringes. The use of reusable syringes under those circumstances increases the risk of infection and spread of blood-borne diseases, particularly when syringes, which have been previously used and are no longer sterile, are used to administer subsequent injections. For example, the World Health Organization (WHO) estimates that blood-borne diseases, such as Hepatitis and human immunodeficiency virus (HIV), are being transmitted due to reuse of such syringes, resulting in the death of more than one million people each year.

Previous attempts at providing single-use or disposable injection devices to remedy such problems in the industry have achieved measurable success but have failed to adequately remedy the existing problems. Pre-filled, single-use injection devices manufactured via injection molding or Form-Fill-Seal (FFS) processes, such as the Uniject™ device available from the Becton, Dickinson and Company of Franklin Lakes, N.J., for example, while offering precise manufacturing tolerances in the range of two thousandths of an inch (0.002-in; 50.8 μm) to four thousandths of an inch (0.004-in; 101.6 μm)—for hole diameters in molded parts, require separate sterilization processes (e.g., gamma radiation) that are not compatible with certain fluids, provide production rates limited to approximately nine thousand (9,000) non-sterile units per hour, and can be provided to an end-user for approximately one dollar and forty cents ($1.40) per dose/unit. Such devices also are not configured to store, mix, and/or deliver a medicament comprising multiple liquids such as a vaccine and an adjuvant.

### SUMMARY

Embodiments of the disclosed subject matter provide systems, assemblies, kits, and methods for medical delivery of liquid agents to a patient. Using a blow-fill-seal (BFS) fabrication technique, a BFS module (such as a vial or other component) is pre-filled with respective fluid (e.g., liquid) agents, which are maintained in separate sealed chambers within the BFS module, for example, until ready for use. In some embodiments, a mixing assembly can be coupled to the BFS module to breach seals of the BFS module and to provide fluid communication between the respective chambers. The liquid agents from the separate chambers can thus be combined prior to administration to a patient. In some embodiments, the combined liquid agents are administered via a needle or cannula that is part of or coupled to a manifold coupled to the BFS module, or via a nozzle that is part of or coupled to a manifold coupled to the BFS module. For example, embodiments of the disclosed subject matter can deliver the combination of the liquid agents from the BFS module as a single dose of a therapeutic agent (e.g., vaccine, drug, medicament, etc.).

In one or more embodiments, a pre-filled medical delivery assembly can comprise a BFS module. The BFS module can have and second longitudinal ends, and can comprise first and second chambers, first and second sealed ports, and first and second actuation members. The first chamber can have a first liquid agent therein, and the second chamber can have a second liquid agent therein. The first and second sealed ports can be proximal to the first longitudinal end of the BFS module. The first sealed port can be in fluid communication with the first chamber, and the second sealed port can be in fluid communication with the second chamber. The first and second actuation members can be proximal to the second longitudinal end of the BFS module. The first actuation member can be in fluid communication with the first chamber, and the second actuation member can be in fluid communication with the second chamber.

In some embodiments, the pre-filled medical delivery assembly can further comprise a mixing assembly constructed to be coupled to the BFS module. The mixing assembly can comprise a base housing, a manifold, and a plunger. The base housing can have third and fourth longitudinal ends, can define a first internal volume extending from the third longitudinal end to the fourth longitudinal end. The manifold can have fifth and sixth longitudinal ends, and can define a second internal volume extending from the fifth longitudinal end to the sixth longitudinal end. The fifth longitudinal end can be disposed proximal to the third longitudinal end of the base housing. A portion of the second internal volume proximal to the fifth longitudinal end can be sized and shaped to act as a mixing chamber for the first and second liquid agents. The sixth longitudinal end can be sized and shaped to receive at least the first longitudinal end of the BFS module therein. The manifold can also comprise first and second longitudinally-extending piercing elements disposed within the second internal volume to align with the first and second sealed ports, respectively. The plunger can comprise a laterally-extending first member and a longitudinally-extending second member. The fourth longitudinal end of the base housing can be sized and shaped to receive the plunger therein.

In some embodiments, upon actuation by a user, the plunger can urge the BFS module and the manifold together to pierce the sealed ports. Upon further actuation by the user, the plunger can compress the actuation members of the BFS module, thereby dispensing the liquid agents from the BFS module and combining together in the mixing chamber of the manifold. In some embodiments, the BFS module and manifold can be removed as a unit from the base housing, for example, by removing the plunger, thereby allowing the actuation members to revert to respective uncompressed states. The combined liquid agents can be administered to a patient (e.g., through a needle or nozzle coupled to or part of the manifold) by re-compressing the actuation members of the BFS module.

In some embodiments, at least the base housing and plunger can be removable from the medical delivery assembly, while at least the manifold remains coupled to the BFS module for administration of the combined liquid agents to a patient. In some embodiments, the base housing and plunger may be reusable with other BFS modules and/or manifolds. In some embodiments, the BFS module may be disposable, for example, after administration of the combined liquid agents to the patient. In some embodiments, both the BFS module and manifold may be disposable.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Where applicable, some elements may be simplified or otherwise not illustrated in order to assist in the illustration and description of underlying features. For example, in some figures, some components have been illustrated using a partial or cutaway view in order to illustrate internal interaction of components. Throughout the figures, like reference numerals denote like elements. An understanding of embodiments described herein and many of the attendant advantages thereof may be readily obtained by reference to the following detailed description when considered with the accompanying drawings, wherein:

FIGS. 1A-1B are simplified schematic diagrams of a pre-filled medical delivery assembly prior to and after breach of sealed ports of a blow-fill-seal (BFS) module, respectively, according to one or more embodiments of the disclosed subject matter;

FIG. 1C is a simplified schematic diagram of the pre-filled medical delivery assembly of FIGS. 1A-1B after further plunger actuation to dispense liquid agents from the BFS module into a mixing chamber, according to one or more embodiments of the disclosed subject matter;

FIG. 1D is a simplified schematic diagram of the pre-filled medical delivery assembly of FIG. 1C removed from the base housing in preparation for administration of the liquid agents to a patient, according to one or more embodiments of the disclosed subject matter;

FIGS. 2A-2C are top, front, and side views, respectively, of an exemplary BFS module, according to one or more embodiments of the disclosed subject matter;

FIGS. 3A-3B are assembled and longitudinally-exploded views, respectively, of an exemplary pre-filled medical delivery assembly, according to one or more embodiments of the disclosed subject matter;

FIGS. 3C-3D are perspective and cross-sectional views, respectively, of the exemplary pre-filled medical delivery assembly of FIGS. 3A-3B in an initial assembled state, according to one or more embodiments of the disclosed subject matter;

FIGS. 4A-4B are perspective and cross-sectional views, respectively, of the exemplary pre-filled medical delivery assembly of FIGS. 3C-3D after first actuation of the plunger to breach sealed ports of the BFS module, according to one or more embodiments of the disclosed subject matter;

FIGS. 5A-5B are perspective and cross-sectional views, respectively, of the exemplary pre-filled medical delivery assembly of FIGS. 4A-4B after second actuation of the plunger to dispense liquid agents into the mixing chamber of the manifold, according to one or more embodiments of the disclosed subject matter;

FIGS. 6A-6B are perspective and cross-sectional views, respectively, of the exemplary pre-filled medical delivery assembly of FIGS. 5A-5B after removal of the plunger, according to one or more embodiments of the disclosed subject matter;

FIGS. 7A-7B are perspective and cross-sectional views, respectively, of the exemplary pre-filled medical delivery assembly of FIGS. 6A-6B after removal of the BFS module and manifold from the base housing, according to one or more embodiments of the disclosed subject matter;

FIGS. 8A-8B are perspective and cross-sectional views, respectively, of the exemplary pre-filled medical delivery assembly of FIGS. 7A-7B after removal of needle cap, according to one or more embodiments of the disclosed subject matter;

FIGS. 9A-9B are perspective and cross-sectional views, respectively, of the exemplary pre-filled medical delivery assembly of FIGS. 8A-8B in preparation for administration, according to one or more embodiments of the disclosed subject matter;

FIGS. 10A-10B are perspective and cross-sectional views, respectively, of the exemplary pre-filled medical delivery assembly of FIGS. 9A-9B during administration of combined liquid agents into a patient, according to one or more embodiments of the disclosed subject matter;

FIGS. 11A-11B are perspective and cross-sectional views, respectively, of the exemplary pre-filled medical delivery assembly of FIGS. 10A-10B after administration, according to one or more embodiments of the disclosed subject matter; and

FIG. 12 is a process flow diagram of an exemplary method for assembly and use of a pre-filled medical delivery assembly for administration of combined liquid agents, according to one or more embodiments of the disclosed subject matter.

### DETAILED DESCRIPTION

### I. Introduction

Described herein are systems, assemblies, kits, and methods for medical delivery of multiple fluid (e.g., liquid) agents (e.g., at least two) to a patient (e.g., human or animal) from a pre-filled (and, in some embodiments, field-assembled or assembled at the point-of-use) module (also referred to herein as a component, bottle, or vial). In some embodiments, at least two liquid agents are sealed within the module and retained separate from each other until a time when it is desirable to mix the liquid agents (e.g., at the time of or prior to administration to the patient). The liquid agents can be any type of agent to be injected into or otherwise delivered to a patient and capable of producing a therapeutic effect, either alone or in combination with an active ingredient. Accordingly, the liquid agents can include, but are not limited to, separate vaccines, drugs, medicaments, diluents, active ingredients, etc. that are desirable to combine for common administration to the patient. For example, in some embodiments, the combination of the liquid agents can form a multi-fluid agent, solution, mixture, suspension, etc. Alternatively or additionally, the liquid agents can be separate components that, when combined, form a vaccine, drug, medicament, etc. For example, in some embodiments, the combination of the liquid agents can comprise a single dose of a therapeutic agent (e.g., vaccine, drug, medicament, etc.). In some embodiments, one or more of the liquid agents in the vial can be tracked, monitored, checked for compatibility, etc., such as by utilization of electronic data storage devices (not shown) coupled to the various modules or components of the delivery system.

In some embodiments, the component may comprise at least one blow-fill-seal (BFS) module that has multiple chambers prefilled with respective liquid agents using a BFS manufacturing technique. At least some of the chambers filled with liquid agents can be sealed from other of the chambers, thereby maintaining the liquid agents separate until combination thereof is desired (e.g., a time for administration to the patient). The BFS module may be constructed, filled, and sealed, according to some embodiments, in a sterile manufacturing environment. BFS modules may, for example, offer a less expensive alternative to typical vials or bottles created via other manufacturing techniques. In some embodiments, BFS modules (e.g., due to the nature of the BFS manufacturing process) may not require separate sterilization (e.g., and may accordingly be compatible with a wider array of liquid agents), may provide enhanced production rates of sterile/aseptic units per hour, and/or may be provided to an end-user for significantly lower per dose/unit costs. In some embodiments, these advantages may come with an attendant drawbacks of reduced manufacturing tolerances and other disadvantages of utilizing a “soft” plastic (e.g., having a Shore/Durometer “D” hardness of between 60 and 70). BFS processes may, for example, offer less precise manufacturing tolerances in the range of five hundredths of an inch (0.05-in; 1.27 mm) to fifteen hundredths of an inch (0.15-in; 3.81 mm)—for linear dimensions, e.g., in accordance with the standard ISO 2768-1 “General tolerances for linear and angular dimensions without individual tolerance indications” published by the International Organization for Standardization (ISO) of Geneva, Switzerland (Nov. 15, 1989), which is incorporated herein by reference.

In some embodiments, a mixing assembly can be coupled to the BFS module to breach seals of the BFS module and to provide fluid communication between the respective chambers to allow mixing of liquid agents external to the BFS module prior to administration. For example, in some embodiments, the combination (e.g., mixing) can occur within a manifold of the mixing assembly. In some embodiments, the combined liquid agents can be administered to a patient via an administration assembly coupled to the manifold or another part of the mixing assembly (e.g., a base housing). In some embodiments, the administration assembly comprises a needle or cannula constructed for subcutaneous, intramuscular, intradermal, or intravenous injection of the combined liquid agents into the patient. Alternatively, in some embodiments, the administration assembly comprises a nozzle. For example, the nozzle can be a spray nozzle that facilitates dispersion of the combined liquid agents into a spray, which configuration may be useful in the administration of the combined liquid agents into a body cavity or orifice (e.g., nasal passage, ear canal, etc.). In another example, the nozzle can be a droplet nozzle that facilitates formation of droplets of the combined liquid agents, which configuration may be useful in the administration of the combined liquid agents to the eyes, for topical application, etc. Alternatively, in some embodiments, the combined liquid agents can be administered without coupling of a separate administration assembly, for example, where a needle, cannula, or nozzle is an integrated part of the mixing assembly (e.g., integrally formed with the manifold).

In some embodiments, the administration assembly and/or the mixing assembly may be configured to be coupled and/or assembled to the BFS module on-site and/or in the field. Alternatively or additionally, in some embodiments, the administration assembly and/or the mixing assembly may be coupled and/or assembled to the BFS module in a manufacturing facility and provided to users as a single, pre-assembled medical device (e.g., with the BFS module contained within a base housing of the mixing assembly but without breach of the sealed ports of the BFS module). The pre-filled multi-liquid medical delivery device may, for example, be capable of delivering combined liquid agents in a controlled manner and without requiring specialized skill in assembling and/or administering delivery of such agent.

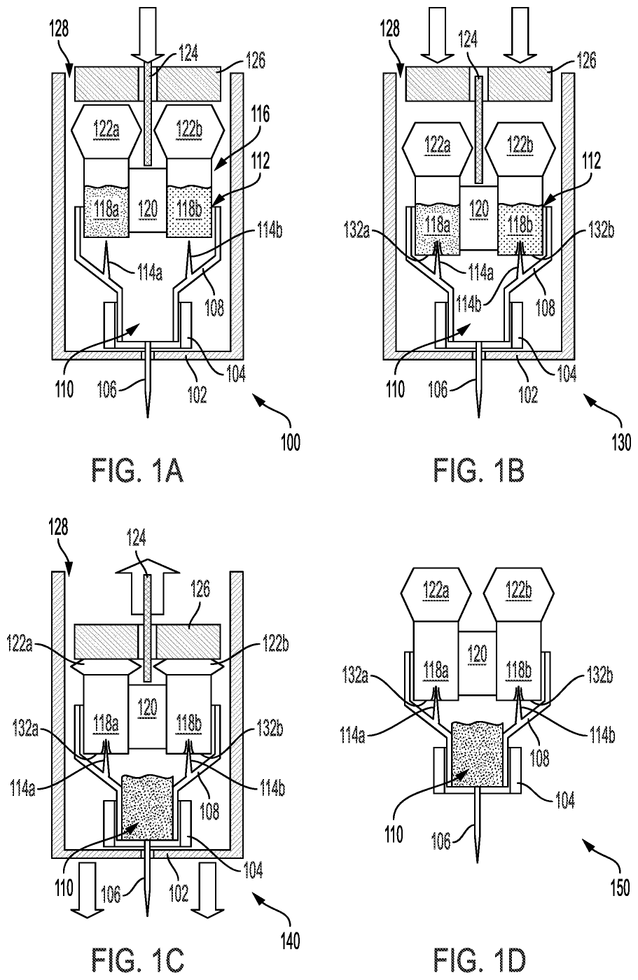

Referring to FIG. 1A, an exemplary pre-filled medical delivery assembly in a first configuration 100 is shown. In some embodiments, the first configuration 100 can be a state of assembly as delivered from a manufacturing facility for use in the field with minimal assembly, or a state of assembly of individual components by a user in preparation for administration. In some embodiments, the medical delivery assembly can include a BFS module 116 and a mixing assembly. The BFS module 116 can include at least two chambers 118a, 118b, each of which has a respective liquid agent sealed therein. The chambers 118a, 118b can be joined together via a bridge member 120 extending laterally between facing surface portions of the chambers. The BFS module 116 can further include at least two actuation members 122a, 122b, for example, air-filled or fluid-filled deformable or compressible chambers in fluid communication with chambers 118a, 118b, respectively. In some embodiments, the actuation members can be capable of independent actuation, such that actuation member 122a can be compressed without requiring compression of actuation member 122b, and vice versa. Compression of the actuation members 122a, 122b can thus exert a pressure on chambers 118a, 118b that urges the contents therein toward port 132a, 132b, which remains sealed in the first configuration 100 of FIG. 1A. In some embodiments, each port 132a, 132b can be sealed by foil, wax, paper, plastic, and/or other thin, pierceable objects or layers coupled to (or formed as part of) the BFS module 116.

In some embodiments, the mixing assembly can comprise a base housing 102, a manifold 108, and a plunger assembly. The base housing 102 can define a first internal volume into which the manifold 108 is disposed, for example, by insertion through an open longitudinal end 128 of the base housing 102. The base housing 102 can include a sidewall and/or laterally-extending surface at an end opposite the open longitudinal end 128 that contacts and/or supports the manifold 108 within the first internal volume. In some embodiments, such a laterally-extending surface of the base housing 102 can include an aperture, for example, through which a portion of an administration member 106, an administration hub 104, and/or the manifold 108 can extend.

The manifold 108 can define a second internal volume into which at least a portion of the BFS module 116 is disposed, for example, by insertion through an open longitudinal end 112 of the manifold 108. The manifold 108 can further include a pair of longitudinally-extending piercing elements 114a, 114b, which can be an integrated part of a sidewall of the manifold 108 or be coupled to the manifold 108 to extend from an internal sidewall thereof. The piercing elements 114a, 114b can be substantially aligned with the sealed ports 132a, 132b of the chambers 118a, 118b of the BFS module 116. In the first configuration 100 of FIG. 1A, the piercing elements can be spaced from, or in non-piercing contact with, ports 132a, 132b, such that the liquid agents remain sealed within the respective chambers 118a, 118b.

A portion of the second internal volume that is distal from the BFS module 116 along a longitudinal direction can act as a mixing chamber 110 for the liquid agents from the BFS module 116. In some embodiments, the mixing chamber 110 can have a cylindrical shape, with a diameter that is substantially constant along the longitudinal direction. A volume of the mixing chamber 110 can be greater than or equal to a volume of the liquid agents contained in chambers 118a, 118b. For example, in some embodiments, the volume of liquid agents in each chamber 118a, 118b is about 0.6 ml, and the volume of the mixing chamber is about 1.25 ml. In some embodiments, the second internal volume of the manifold 108 can have a funneled portion connecting to a top end of the mixing chamber 110. The funneled portion can have a frustoconical or tapered shape in cross-section that narrows toward the mixing chamber 110. In some embodiments, each piercing element 114a, 114b is disposed on or extends from an inclined internal sidewall of the manifold 108 that defines the tapered shape of the funneled portion.

In some embodiments, the second internal volume of the manifold 108 can further have a receiving portion connecting to a top end of the funneled portion, e.g., extending from the longitudinal end 112 of the manifold 108 to the funneled portion. In some embodiments, an inner surface of the receiving portion may have a size and shape that matches that of an outer perimeter of at least a portion of the BFS module 116, e.g., the first and second chambers 118a, 118b. The receiving portion of the second internal volume of the manifold 108 may thus have a maximum cross-sectional dimension that is greater than a diameter of the mixing chamber 110.

In some embodiments, the medical delivery assembly can further include an administration assembly, which can include an administration member 106 coupled to an administration hub 104. In some embodiments, the administration hub 104 is coupled to (e.g., screwed onto) or integrated with the manifold 108 (e.g., at a distal end of the mixing chamber 110). In some embodiments, the administration member 106 may include a needle for at least one of subcutaneous, intramuscular, intradermal, and intravenous injection of the fluid agent into the patient. For example, the needle can have a length of 0.5 mm to 4 mm, inclusive, or in a range of 4 mm to 15 mm, inclusive, or in a range of 15 mm to 30 mm, inclusive, depending on the desired manner of injection.

For ease of explanation and description, the figures and the description herein generally refer to the administration member as a needle. However, it should be noted that, in other embodiments, the administration member 106 may include a nozzle (not shown) configured to control administration of the fluid agent to the patient. The nozzle may include a spray nozzle, for example, configured to facilitate dispersion of the fluid agent into a spray. Accordingly, a hub 104 fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, a hub 104 including a droplet nozzle may be useful in the administration of a fluid agent by way of droplets, such as administration to the eyes, topical administration, and the like.

In some embodiments, the plunger assembly can include a plunger first member 126 and a plunger second member 124. The plunger first member 126 can extend in a lateral direction, and the plunger second member 124 can extend in the longitudinal direction. In some embodiments, the plunger second member 124 can move along the longitudinal direction at least partially independently of the plunger first member 126, for example, by extending through a slot or gap in the plunger first member 126, as shown in FIG. 1A. The plunger assembly can be inserted into and/or coupled to the base housing 102 via the open longitudinal end 128 thereof. In the first configuration 100 of FIG. 1A, the plunger first member 126 and/or plunger second member 124 can be spaced from the BFS module 116, or subtle contact with the BFS module 116 so as not to compress the actuation members 122a, 122b or to push the piercing elements 114a, 114b through the respective sealed ports 132a, 132b. A bottom surface of the plunger first member 126 can thus face or be in non-compressive contact with the actuation members 122a, 122b, while at least a portion of the plunger second member 124 extends between the actuation members 122a, 122b, for example, to contact and push on bridge member 120 to transition the assembly from the first configuration 100 to the second configuration 130 of FIG. 1B.

To transition the assembly from the first configuration 100 of FIG. 1A to the second configuration 130 of FIG. 1B, the plunger second member 124 can be pushed by the user along the longitudinal direction toward the manifold 108. A longitudinal end of the plunger second member 124 abuts the bridge member 120 and forces the BFS module 116 downward into contact with the piercing elements 114a, 114b, such that piercing elements breach the sealed ports 132a, 132b of the liquid-agent-containing chambers 118a, 118b. The chambers 118a, 118b are thus brought into fluid communication with the second internal volume of the manifold 108 via the breached ports 132a, 132b, as shown in FIG. 1B.

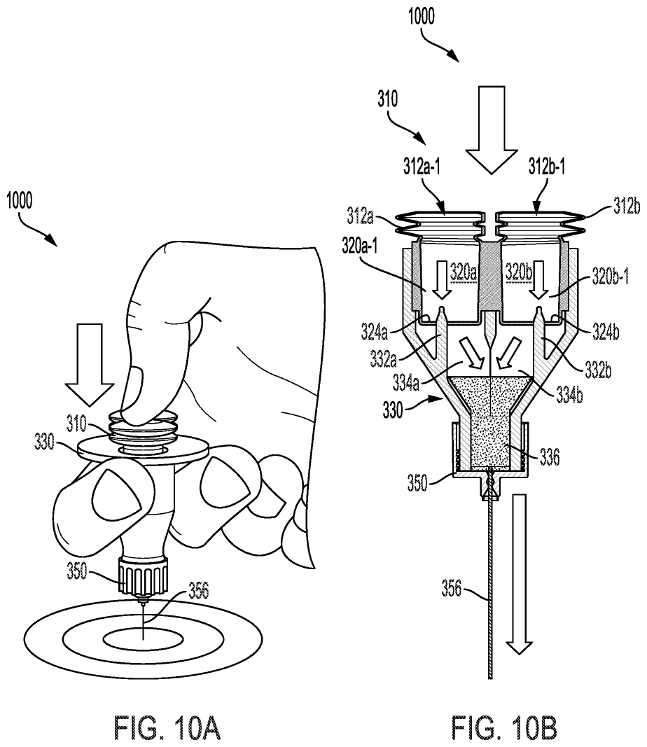

The plunger first member 126 can then be pushed by the user along the longitudinal direction toward the manifold 108, for example, by screwing the plunger first member 126 into a top threaded end of the base housing 102, such that a bottom surface of the plunger first member 126 abuts the top end of the BFS module and compresses the actuation members 122a, 122b, thereby forcing the contents out of the chambers 118a, 118b. For example, the liquid agents from each chamber 118a, 118b can flow around the piercing elements 114a, 114b extending through the ports 132a, 132b and into the funneled portion of the manifold 108. The funneled portion then directs the liquid agents into the mixing chamber 110, where the liquid agents mix to form a desired combination for a fluid or drug agent to be administered, as shown by the third configuration 140 of FIG. 10.

After mixing, the assembly can be converted to the fourth configuration 150 of FIG. 1D for administration of the combined liquid agents to a patient, for example, by removing the BFS module 116 coupled to the manifold 108 from the base housing 102. The plunger first member 126 and the plunger second member 124 can be removed from longitudinal end 128 of the base housing 102, which allows actuation members 122a, 122b to revert to their original uncompressed states. In some embodiments, the BFS module 116, the manifold 108, and the administration assembly can be removed from the base housing 102 as a single unit, for example, via longitudinal displacement through the open longitudinal end 128 of the base housing 102. In the configuration 150 of FIG. 1D, the contents of the mixing chamber 110 can be dispensed via the administration member 106 by re-compressing the first and second actuation members 122a, 122b.

As generally understood, the fluid or drug agent may include any type of agent to be injected into a patient (e.g., mammal, either human or non-human) and capable of producing an effect (alone, or in combination with an active ingredient). Accordingly, the agent may include, but is not limited to, a vaccine, a drug, a therapeutic agent, a medicament, a diluent, and/or the like. According to some embodiment, either or both of the fluid agent and the active ingredient (i.e., the drug agent and/or components thereof) may be tracked, monitored, checked for compatibility with each other, etc., such as by utilization of electronic data storage devices (not shown) coupled to the various modules or components of the pre-filled multi-liquid medical delivery assembly, such as the BFS module 116, the hub 104, the manifold 108, and/or the base housing 102.

According to some embodiments, manifold 108, the hub 104, and/or the housing 102 may be composed of a medical grade material. In some embodiments, the manifold 108, the hub 104, the housing 102, a cap for the administration member 106, the plunger first member 126, and/or the plunger second member 124 may be composed of a thermoplastic polymer or other relatively hard plastic (e.g., greater than 80 on the Rockwell “R” scale), such as, but not limited to, polybenzimidazole, acrylonitrile butadiene styrene (ABS), polystyrene, polyvinyl chloride, or the like. In some embodiments, at least the base housing and/or the plunger are formed of a material having a hardness greater than that of the manifold. For example, the base housing, the plunger, or both can be formed of polypropylene (e.g., high-density polypropylene), while the manifold can be formed of polycarbonate.

In some embodiments, the pre-filled multi-liquid medical delivery assembly may be advantageously manufactured (in mass quantities) in separate parts or portions, namely, at least the relatively soft plastic BFS module 116 portion (e.g., a “first” piece), the relatively hard plastic manifold (e.g., a “second” piece), the hub 104, administration member 106, and corresponding cap for the administration member (e.g., a “third” piece), and/or the base housing 102, plunger first member 126, and plunger second member 124 (e.g., a “fourth” piece) with such different plastic parts/portions being selectively coupled to administer a medication (e.g., of at least two fluids) to a patient. In some embodiments, the BFS module 116 may be specially designed and/or configured to separately store or house the at least two different fluids until such time as the pre-filled multi-liquid medical delivery assembly is activated (e.g., by breaching the sealed ports, dispensing the liquid agents from the BFS vial into the mixing chamber of the manifold, and subsequently injecting the mixed liquid agents into the patient).

In some embodiments, the pre-filled medical delivery system or assembly may comprise various inter-connected and/or modular components. In some embodiments, some or all of the various components can be provided in an unassembled state as a kit, for example, for assembly in the field and/or at a time just prior to administration. In some embodiments, the pre-filled medical delivery assembly, or the kit for forming the medical delivery assembly, can include additional components beyond those specifically illustrated in FIGS. 1A-1D. In some embodiments, the components of the kit, e.g., the BFS module 116 and the mixing assembly (e.g., base housing 102, manifold 108, plunger first member 126, and plunger second member 124) can be manufactured, packaged, shipped, stored, and/or provided as separate components. In such a manner, the mixing assembly may not need to be stored or shipped in accordance with often restrictive requirements imposed on medicaments and may accordingly reduce the amount of space required for such specialized storage and/or shipping. The mixing assembly may also or alternatively be manufactured, stored, and/or shipped in advance (e.g., at a first time) while the BFS module 116 that is pre-filled with the liquid agents may be manufactured, stored, and/or shipped at a later time (e.g., a second time). In some embodiments, the delay between the first time and the second time may be lengthy without causing detrimental effects, as the mixing assembly may be stored, in some embodiments, indefinitely. In such a manner, units of the mixing assembly may be provided to be on-hand in advance of the availability and/or arrival of the BFS module 116, reducing supply chain constraints in the case of proactive mixing assembly procurement. Alternatively or additionally, at least some components of the mixing assembly may be reusable with other BFS modules, for example, the base housing 102, the plunger first member 126, and/or the plunger second member 124. Thus, unassembled components of pre-filled medical delivery system kits according to embodiments of the disclosed subject matter need not be provided together in the same package or at the same time.

In some embodiments, fewer or more components 102-128 and/or various configurations of the depicted components 102-128 may be included in the pre-filled multi-liquid medical delivery assembly without deviating from the scope of embodiments described herein. In some embodiments, the components 102-128 may be similar in configuration and/or functionality to similarly named and/or numbered components as described herein.

### II. Multi-Fluid Blow-Fill-Seal (BFS) Modules

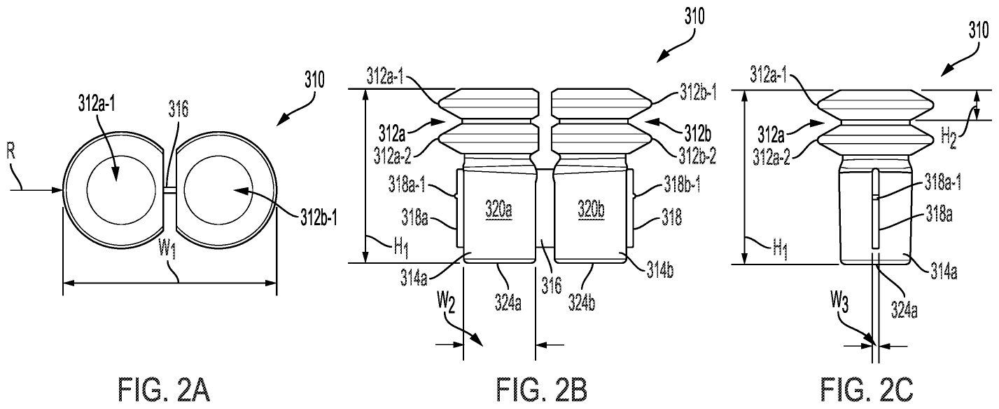

Referring to FIGS. 2A-2C, an exemplary multi-fluid BFS module 310 according to some embodiments is shown. In the illustrated example, the multi-fluid BFS module 310 comprises a plurality of collapsible portions 312a, 312b that are arranged, configured, disposed, and/or coupled to exert pressure when axially compressed (e.g., in a longitudinal direction substantially parallel to module height, H1, and/or, portion height, H2). Each collapsible portion 312a, 312b may comprise, for example, a plurality of bellows sections, for example respective pairs of bellow 312a-1, 312a-2 and bellows 312b-1, 312b-2. For example, each bellows section can have a height, H2, along a longitudinal direction of BFS module 310 of 4.2 mm. In addition, each bellows section may have a substantially circular geometry in a top down view, for example, defining a radius, R, of 7.4 mm. The bellows sections can be constructed and arranged to collapse upon each other in the case that an axial compressive force not depicted) is received thereby. In some embodiments, the collapsible portions of the BFS module 310 can define a maximum width of the module in a lateral direction. For example, the collapsible portion can define a width, W₁, of 27.3 mm.

In some embodiments, the multi-fluid BFS module 310 may comprise a plurality of body portions 314a, 314b connected or joined together via a web feature or bridge member 316. For example, each body portion 314a, 314b can have a width, W₂, along the lateral direction of 9.4 mm. In some embodiments, the body portion 314a, 314b may have a substantially cylindrical shape, such that the lateral width, W₂, corresponds to the diameter of the cylinder. In some embodiments, the body portions may constitute the majority of the module height along the longitudinal direction. For example, the BFS module 310 can have an overall height, H1, along the longitudinal direction of 22.3 mm, with the pair of bellows occupying ˜8.4 mm at the top of the module. In the illustrated example, each body portion 314a, 314b may comprise and/or define an exterior rib or flange 318a-b that extends longitudinally along opposing side portions of the BFS module 310. For example, each flange 318a, 318b can have a width, W₃, in a transverse direction of 0.8 mm. In some embodiments, each flange can have a substantially square cross-sectional geometry, such that W₃ also represents the width of the flange in the lateral direction. In some embodiments, each flange 318a, 318b may respectively comprise or define a rib, nub, or protrusion 318a-1, 318b-1 that extends laterally outward from the outer extents of the respective flange 318a, 318b.

Each body portion 314a, 314b may, for example, define one or more of the fluid reservoirs 320a, 320b. In some embodiments, each fluid reservoir 320a, 320b may be in fluid communication with a respective one of the collapsible portions 312a, 312b at a first end (e.g., top end) thereof. In some embodiments, the collapsible portions 312a, 312b may be coupled to convey fluid pressure (in the case that force is received) to respectively coupled fluid reservoirs 320a, 320b. Alternatively or additionally, each fluid reservoir 320a, 320b may have a respective sealed port 324a, 324b at a second opposing end (e.g., bottom end) thereof. In some embodiments, the seals of the ports comprises a foil, wax, paper, a section of the BFS module (e.g., a bottom wall of the BFS module), or any combination of the foregoing. In some embodiments, the seals of the ports 324a, 324b are breached, for example, by puncturing, piercing, rupturing, penetrating, or otherwise breaking the seal, in order to access and/or dispense contents of the fluid reservoirs 320a, 320b.

In some embodiments, fewer or more components 312-324 and/or various configurations of the depicted components 312-324 may be included in a multi-fluid BFS module without deviating from the scope of embodiments described herein. In some embodiments, the components 312-324 may be similar in configuration and/or functionality to similarly named and/or numbered components as described herein.

### III. Pre-Filled Multi-Fluid Medical Delivery Assemblies

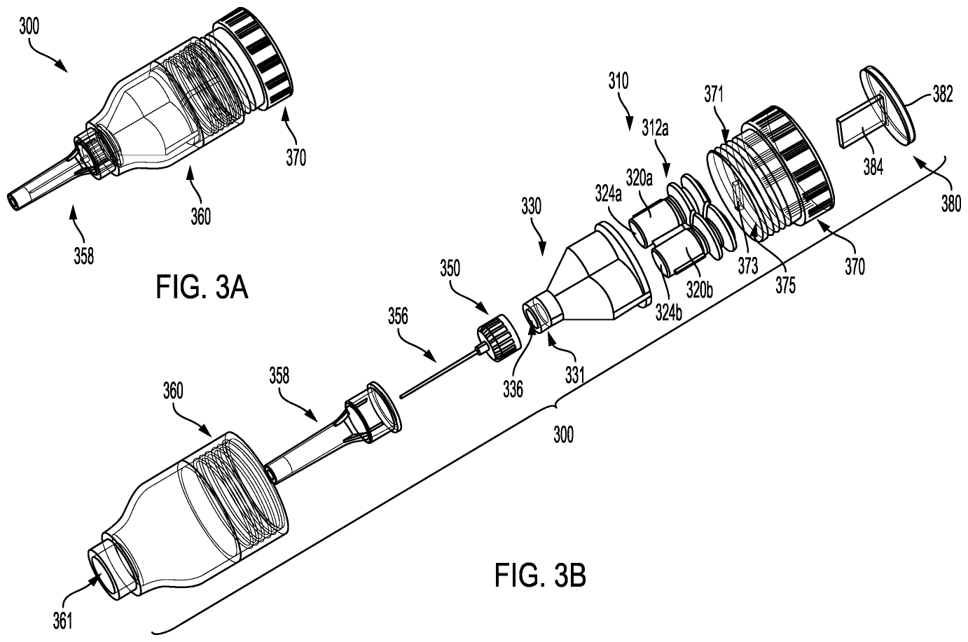

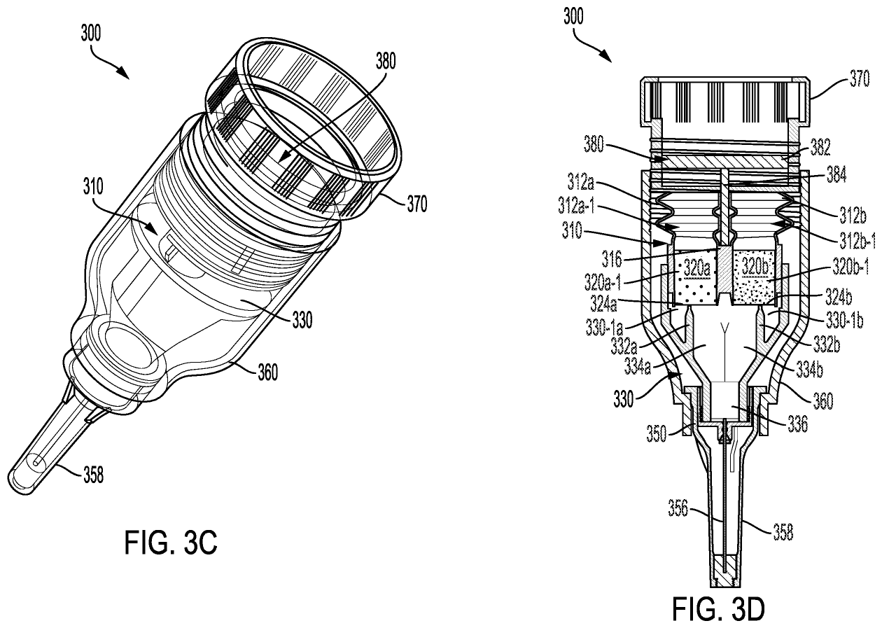

Referring to FIGS. 3A-3D, an exemplary pre-filled multi-liquid medical delivery assembly 300 according to some embodiments are shown. In the illustrated example, the pre-filled multi-liquid medical delivery assembly 300 comprises a BFS module 310, an administration assembly, and a mixing assembly. The BFS module 310 comprises and/or defines a plurality of collapsible bellows 312a, 312b, each of which can be axially aligned (e.g., along a longitudinal direction) with a corresponding fluid reservoir 320a, 320b. The administration assembly comprises an administration member 356 (e.g., a needle) and a hub 350 that couples to and/or houses the administration member 356. The mixing assembly comprises a manifold 330 (also referred to as a mixing connector), a base housing 360 (also referred to as a “hard pack,” outer casing, or housing), and a plunger assembly (e.g., including end cap 370 (also referred to as a first member of the plunger) and a removable plunger disk 380 (also referred to as a second member of the plunger)). In the illustrated example, the end cap 370 can engage with the base housing 360 via a threaded connection. The plunger disk 380 can have a protruding portion 384 that extends from a disk portion 382 and is sized and shaped to fit into a corresponding through-slot 373 of the end cap 370, thereby allowing the plunger disk 380 to move longitudinally with respect to the end cap 370 to interact with the BFS module 310.

In some embodiments, the fluid reservoirs 320a, 320b may be filled (fully or partially) with a fluid or other agent to be delivered, e.g., to a patient. According to some embodiments, each fluid reservoir 320a, 320b may be filled with a different fluid such as a different liquid or gas, such different fluids agents being combined, introduced, and/or mixed in the pre-filled multi-liquid medical delivery assembly 300 (e.g., via the manifold 330 thereof) to define a fluid agent for delivery to the patient. In some embodiments, a first fluid reservoir 320a may house or contain a vaccine, for example, while a second fluid reservoir 320b may house or contain an adjuvant (and/or carrier fluid, catalyst, diluent, etc.). According to some embodiments, any or all of the fluids may be injected into the BFS module 310 (e.g., into respective fluid reservoirs 320a, 320b thereof) in a sterile environment during manufacture via a BFS process and sealed within the BFS module 310 via the fluid seals 324a, 324b. The fluid seals 324a, 324b may comprise portions of the molded BFS module 310, for example, that are configured to be pierced to expel the respective fluids, e.g., such as by providing a flat or planar piercing surface and/or by being oriented normal to an axis of the BFS module 310 (and/or the pre-filled multi-liquid medical delivery assembly 300). In some embodiments, the fluid seals 324a, 324b may comprise foil, wax, paper, plastic, and/or other thin, pierceable objects or layers coupled to (or formed as part of) the BFS module 310.

According to some embodiments, the manifold 330 may be axially engaged to couple with the BFS module 310 via application of a mating axial force. The fluid reservoirs 320a, 320b may be urged into the manifold 330, for example, such that cooperatively shaped interior chambers and/or grooves (not shown) accept the fluid reservoirs 320a-b, thereby removably coupling the BFS module 310 and the manifold 330. In some embodiments, the interior chambers and/or grooves (and/or other interior features) and/or the fluid reservoirs 320a, 320b may be shaped such that uncoupling of the BFS module 310 and the manifold 330 is mechanically prohibited, or at least resisted. According to some embodiments, insertion of the fluid reservoirs 320a, 320b into the manifold 330 and/or otherwise mating thereof may cause the bellows 312a, 312b to remain outside of and/or axially adjacent to the manifold 330.

In some embodiments, the hub 350 may be coupled to the manifold 330, such as via a threaded connection. The hub 350 may comprise internal threads (not shown) that correspond and cooperate with external threads of the manifold 330 such that they may be rotationally and/or removably coupled. According to some embodiments the hub 350 may couple to and/or retain the administration member 356. The administration member 356 may be inserted into and/or through the hub 350, for example, such that it comprises a first or administration end extending axially distal from the manifold 330 and a second end (not shown) disposed within the hub 350 and/or extending into the manifold 330. In some embodiments, the administration end and/or a distal portion of the administration member 356 may be housed, shrouded, and/or covered by the cap 358. According to some embodiments, the cap 358 may be configured to house the administration member 356 and to removably couple to the hub 350 (e.g., by fitting over an external portion thereof).

According to some embodiments, the manifold 330 and hub 350 combination may be utilized to couple and/or mate the administration member 356 with the BFS module 310 to provide a mechanism via which the administration member 356 may be coupled to the relatively soft plastic of the BFS module 310 in a reliable manner. Due to the nature of the BFS plastic and/or process and/or the small form-factor of the BFS module 310, for example, providing external threads directly on the BFS module 310 would not be a viable option, as it would result in an imprecise, unreliable, and/or non-water tight coupling (e.g., the threads would be deformable even if they could be properly manufactured to within the desired tolerances, which itself is not a likely result) between the BFS module and the hub. Indeed, the “soft” plastics required for the BFS process would be susceptible to heat deformation of machined features during formation attempts as well as deformation due to mechanical stress during utilization, thus making a threaded coupling of hub 350 directly to the BFS module 310 impractical.

In some embodiments, the pre-filled multi-liquid medical delivery assembly 300 may utilize and/or employ a BFS module 310 to safely, inexpensively, reliably, and/or conveniently administer a multi-fluid medicament to a patient or other target (not shown). The BFS module 310 may comprise, for example, a plurality of collapsible sections or “bellows” 312a-b that each define a respective interior bellows volume 312a-1, 312b-1. According to some embodiments, the bellows 312a-b may define two or more adjacently disposed portions of the BFS module 310 that are connected via a web or bridge 316. In some embodiments, each of the two portions of the BFS module 310 may comprise and/or define a fluid reservoir 320a-b that defines a respective reservoir volume 320a-1, 320b-1 that is in communication with the respective and/or aligned interior bellows volume 312a-1, 312b-1. In some embodiments, each connected reservoir volume 320a-1, 320b-1 and interior bellows volume 312a-1, 312b-1 pair may house and/or contain a combination of liquid and gaseous components. When disposed in a “downward” configuration as depicted in FIG. 3D, for example, the liquid components may rest in the respective fluid reservoirs 320a-b (e.g., substantially or entirely in the reservoir volumes 320a-1, 320b-1) against respective seals 324a-b thereof and the gaseous components may be substantially disposed in the respective interior bellows volumes 312a-1, 312b-1. In some embodiments, a first liquid in a first reservoir 320a may comprise approximately six tenths of a milliliter (0.6 ml) of an adjuvant and/or a second liquid in a second reservoir 320b may comprise approximately six tenths of a milliliter (0.6 ml) of vaccine or other active ingredient.

According to some embodiments, the pre-filled multi-liquid medical delivery assembly 300 may comprise a mixing connector or manifold 330 that comprises and/or defines a chamber 330-1a, 330-1b configured to receive and/or mate with one or more of the reservoirs 320a-b. In some embodiments, and as depicted in a first state or configuration in FIGS. 3C-3D, the fluid reservoirs 320a-b may be disposed in the respective chambers 330-1a, 330-1b of the manifold 330. According to some embodiments, the manifold 330 may comprise a plurality of piercing elements 332a-b disposed adjacent to the respective seals 324a-b of the reservoirs 320a-b. In the first state, for example, the seals 324a-b may rest upon the tips of the piercing elements 332a-b. In some embodiments, the manifold 330 may comprise a dispensing chamber 334a-b for each respective reservoir 320a-b. According to some embodiments, one or more of the piercing elements 332a-b may be disposed within each of the dispensing chambers 334a-b.

In some embodiments, the manifold 330 may comprise and/or define an outlet port 336 in fluid communication with the dispensing chambers 334a-b. The outlet port 336 may be disposed, for example, in a neck portion of the manifold 330 that comprises external threads thereon. According to some embodiments, a needle hub 350 may be coupled to the manifold 330. Interior threads of the needle hub 350 may correspond to and/or mate with the external threads of the neck of the manifold 330, for example, permitting selective coupling therebetween. In some embodiments, the needle hub 350 may comprise a needle 356 (and/or other and administration member) and/or a cap 358 that protects and/or shields at least a distal end of the needle 356.

According to some embodiments, the pre-filled multi-liquid medical delivery assembly 300 may comprise an outer housing 360. The outer housing 360 may be constructed of hard and/or durable plastic or other materials, for example, and may shield and/or protect the BFS module 310, manifold 330, and/or needle hub 350. In some embodiments, the outer housing 360 may selectively couple to an end cap 370. The end cap 370 and the outer housing 360 may, as depicted for example, comprise corresponding threads that permit engagement or disengagement of the end cap 370 with the outer housing 360. In some embodiments, the end cap 370 may house, retain, accept, be coupled to, and/or comprise a plunger 380. The plunger 380 may comprise, for example, a radial disk portion 382 that seats within the end cap 370 and/or a plunger portion 384 that extends axially toward the BFS module 310. In some embodiments, the end cap 370 (also referred to herein as plunger first member) and the plunger 380 (also referred to herein as plunger second member) may be considered components of a plunger assembly.

According to some embodiments, the pre-filled multi-liquid medical delivery assembly 300 may include a modular design of separately constructed components 310, 330, 350, 360, 370, and 380 cooperatively arranged and coupled to one another. Alternatively or additionally, in some embodiments one or more of the separately illustrated components can be combined together into an integrated component (e.g., formed together rather than formed separately and subsequently coupled together). For example, in some embodiments, the hub 350 may an integrated part of manifold 330. In some embodiments, at least the base housing 360, end cap 370, and plunger disk 380 can be removable from the medical delivery assembly 300, while at least the manifold 330 can remain coupled to the BFS module 310 for administration of combined liquid agents to a patient. In some embodiments, the base housing 360, end cap 370, and/or plunger disk 380 may be reusable with other BFS modules and/or manifolds. In some embodiments, the BFS module 310 may be disposable, for example, after administration of combined liquid agents to the patient. In some embodiments, both the BFS module 310 and manifold 330 may be disposable.

In some embodiments, and referring again to the first state depicted in FIGS. 3C-3D, the needle hub 350 may be coupled to the manifold 330, with the combination being seated in and/or housed by the outer housing 360. Also in the first state, the BFS module may be seated and/or mated with the manifold 330 and the end cap 370 may be partially engaged and/or mated with the outer housing 360. In this configuration and/or state, the plunger portion 384 of the plunger 380 may be disposed adjacent to and/or resting upon or against the bridge 316. In some embodiments, this first state may comprise an assembled consumer and/or end-user (e.g., healthcare worker) state of the pre-filled multi-liquid medical delivery assembly 300 that represents a configuration in which it is provided for end use.

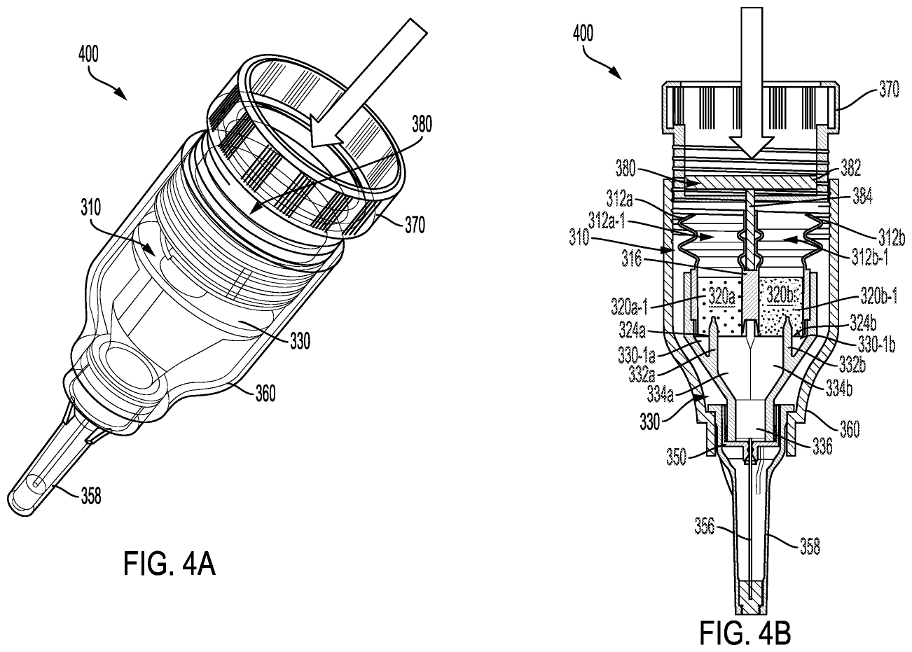

According to some embodiments, a second state 400 of the pre-filled multi-liquid medical delivery assembly may be depicted in FIGS. 4A-4B. In the second state 400, the plunger 380 has been pressed axially downward (or inward), as represented by the arrows. Force applied to the radial disk portion 328 of the plunger 380 may cause, for example, the plunger portion 384 to engage with the bridge member 316 and push the BFS module 310 downward and/or longitudinally (e.g., axially) further into the manifold 330. Such downward movement may, as depicted, cause the piercing elements 332a-b to engage with and pierce or rupture the seals 324a-b. A user (not shown) may accomplish this piercing by, for example, urging their thumb down upon the plunger 380 while holding the pre-filled multi-liquid medical delivery assembly 300 in their hand (e.g., single-hand operation may be possible). In some embodiments, protrusion 318a-1 and/or protrusion 318b-1 of the BFS module 310 can engage with or be received in respective sidewall recesses of the manifold 330 to provide an indication (e.g., an audible or tactile click) of an acceptable displacement induced by pushing the plunger disk and/or to resist withdrawal of the BFS module 310 after pushing by the plunger disk 380.

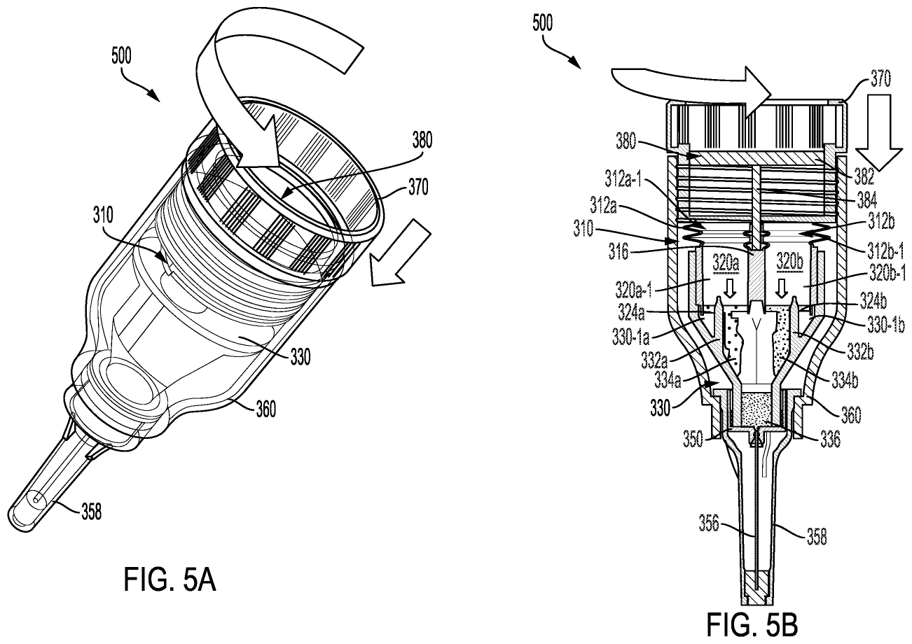

In some embodiments, once the seals 324a-b have been pierced, the pre-filled multi-liquid medical delivery assembly may be advanced to a third state 500 as depicted in FIGS. 5A-5b. In the third state 500, for example, the user may advance the threads of the end cap 370 to fully engage and/or seat the end cap 370 with the outer housing 360. According to some embodiments, as the bottom of the end cap 370 is advanced into the outer housing 360 it may engage with and compress the bellows 312a-b, thereby forcing the gaseous components into the reservoirs 320a-b and thereby forcing the liquid components (at least partially) through the breaches in the seals 324a and into the respective dispensing chambers 334a-b. According to some embodiments, the cross-sections of the piercing elements 332a-b may be configured to permit the fluids/liquids to enter the dispensing chambers 334a-b. The piercing elements 332a-b may, for example, comprise an “x” cross-section defining four (4) axial channels for fluid flow through the breaches. The dispensing chambers 334a-b may, in some embodiments, comprise a single mixing chamber that permits unfettered mixing and/or combining of the multiple fluid/liquid agents. In some embodiments, the dispensing chambers 334a-b may comprise various baffles, weirs, and/or other hydraulic features that partially separate the dispensing chambers 334a-b and/or provide for specific and/or enhanced mixing therein. According to some embodiments, as the dispensing chambers 334a-b may contain air or another gaseous component, the entering liquids/fluids may mix with such existing component(s) in the dispensing chambers 334a-b (and/or in the outlet port 336). In some embodiments, because the combined volumes of the compressed bellows 312a-b, the reservoirs 320a-b, and the dispensing chambers 334a-b (and/or in the outlet port 336) is smaller than in the previous states, one or more of the gaseous components may become at least partially compressed.

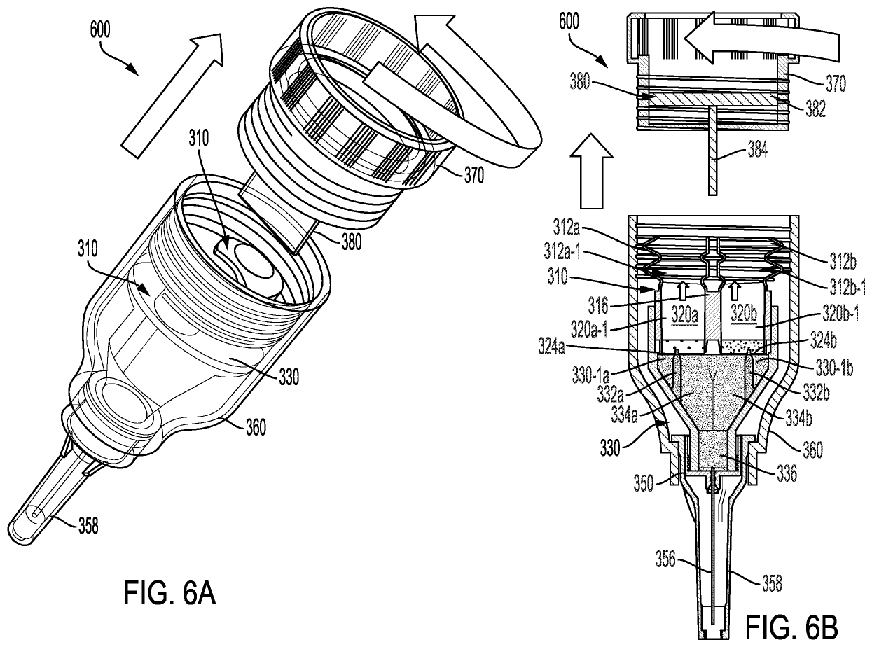

According to some embodiments, once the fluids/liquids have been introduced (e.g., at the third state 500) the pre-filled multi-liquid medical delivery assembly may be advanced to a fourth state 600 as depicted in FIGS. 6A-6B. In the fourth state 600, for example, the end cap 370 may be disengaged from the outer housing 360 (e.g., by fully reversing the threaded connection therebetween). In some embodiments, the plunger 380 may be retained by the end cap 370 and may accordingly also be removed from the outer housing 360 and/or from the components housed therein. According to some embodiments, upon removal of the end cap 370 the bellows 312a-b may spring back to their uncompressed positions, thereby increasing the combined interior volumes of the bellows 312a-b, the reservoirs 320a-b, and the dispensing chambers 334a-b (and/or in the outlet port 336). In some embodiments, this expansion may permit any gaseous components originally housed in the dispensing chambers 334a-b to advance upward into the reservoirs 320a-b and/or into the bellows 312a-b. In such a manner, for example, all of the liquid components from the reservoirs 320a-b may now be fully mixed or introduced in the dispensing chambers 334a-b (and/or the outlet port 336).

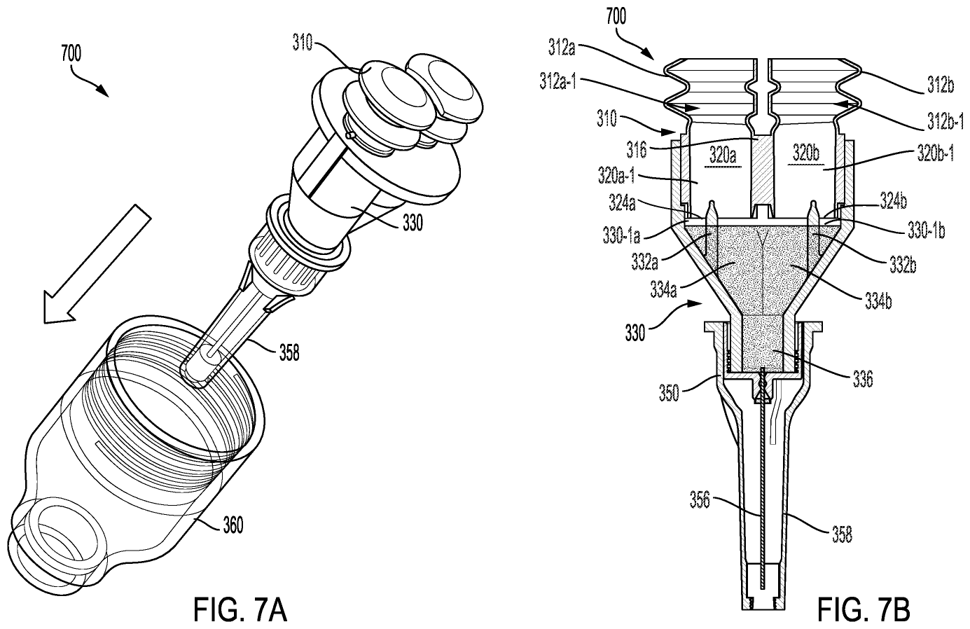

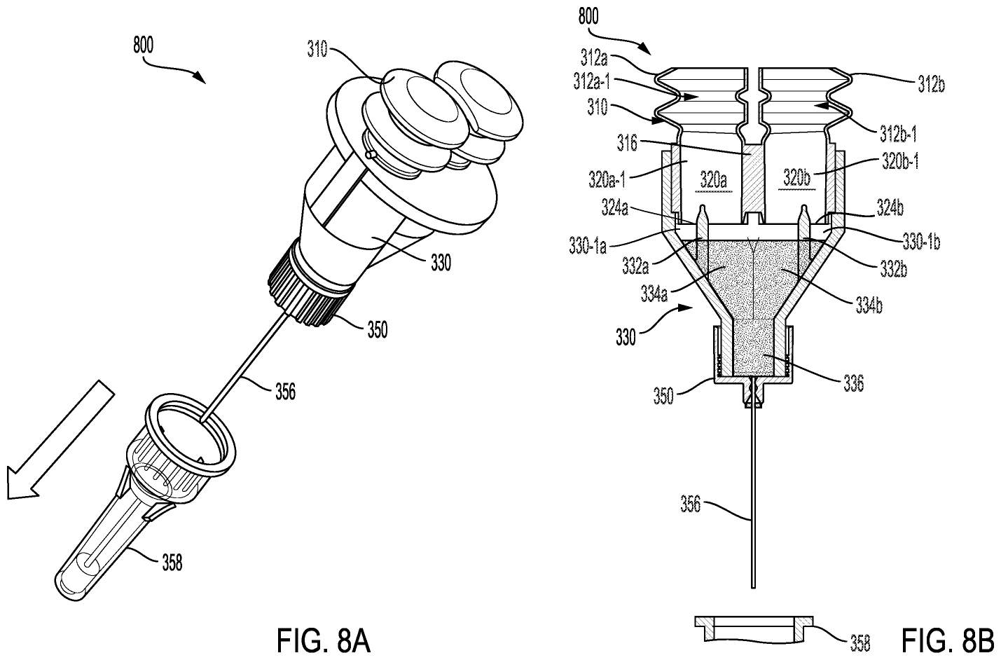

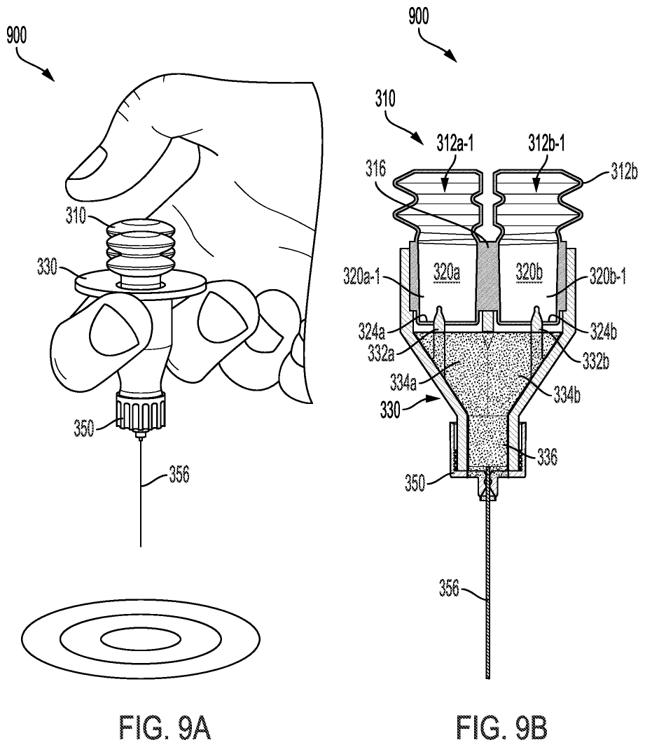

In some embodiments, the pre-filled multi-liquid medical delivery assembly may be advanced to a fifth state 700 as depicted in FIGS. 7A-7B. In the fifth state 700, for example, the combined needle hub 350, manifold 330, and BFS module 310 may be removed from the outer housing 360. According to some embodiments, the pre-filled multi-liquid medical delivery assembly may be advanced to a sixth state 800 as depicted in FIGS. 8A-8B. In the sixth state 800, for example, the combined needle hub 350, manifold 330, and BFS module 310 may be prepared for injection/administration. As depicted in FIGS. 8A-8B, for example, the cap 358 may be removed to expose the administration or outlet end of the needle 356. In some embodiments, the pre-filled multi-liquid medical delivery assembly may be advanced to a seventh state 900 as depicted in FIGS. 9A-9B. In the seventh state 900, for example, the combined needle hub 350, manifold 330, and BFS module 310 may be grasped (e.g., single-handedly as depicted) and positioned over a target (e.g., a chosen administration site of a patient).

According to some embodiments, the pre-filled multi-liquid medical delivery assembly may be advanced to an eighth state 1000 as depicted in FIGS. 10A-10B. In the eighth state 1000, for example, the combined needle hub 350, manifold 330, and BFS module 310 may be engaged to administer the combined fluid agent/medicament to the patient. The needle 356 (and/or other administration member) may be inserted into the target, for example, and the bellows 312a-b may be compressed (via application of an axially downward force; e.g., by the user's thumb as depicted). In some embodiments, compression of the bellows 312a-b may force any gaseous components therein to expel the multi-liquid agent through the needle 356 and into the target. According to some embodiments, and as utilized for purposes of non-limiting example herein, each liquid agent may be provided in an approximately six tenths of a milliliter (0.6 ml) actual volume such that an estimated one tenth of a milliliter (0.1 ml) of waste or loss (each) may be expected to be retained within the pre-filled multi-liquid medical delivery assembly 300, resulting in an effective dosage of one milliliter (1 ml)—e.g., five tenths of a milliliter (0.5 ml) of each of the liquids in the case of two (2) liquid agents. In some embodiments, the liquid (or other fluid) agents may be provided in disparate volumes such as by altering the sizes and/or geometries of the reservoirs 320a-b.

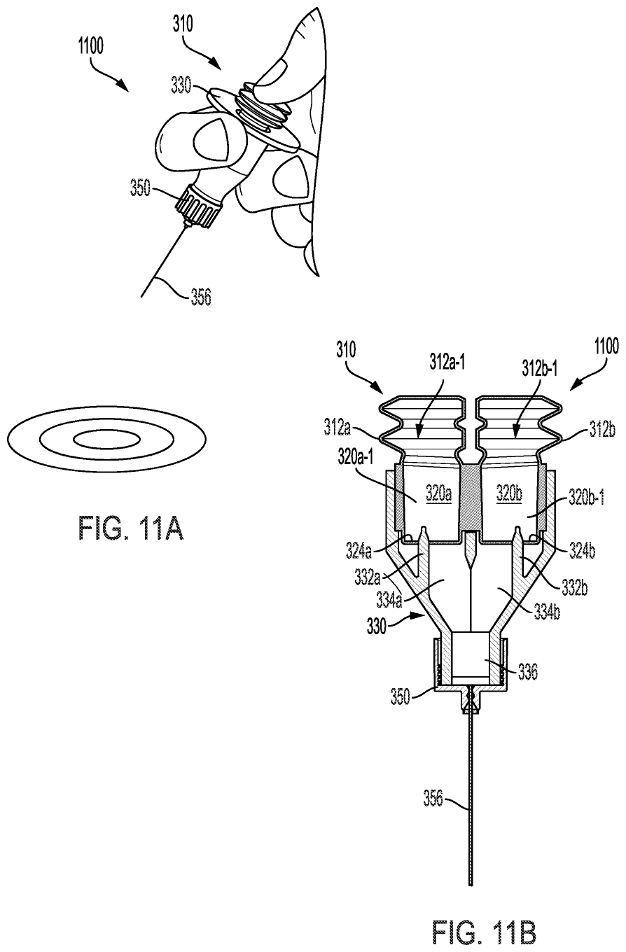

In some embodiments, the pre-filled multi-liquid medical delivery assembly may be advanced to a ninth state 1100 as depicted in FIGS. 11A-11B. In the ninth state 1100, for example, the combined needle hub 350, manifold 330, and BFS module 310 may be removed from the patient, such as by backing the needle 356 out of the target site. According to some embodiments, it may be desirable to maintain pressure upon the bellows 312a-b to maintain the bellows 312a-b in their compressed state until the needle 356 has been removed from the target, such as to prevent application of suction forces to the target. Upon removal, and as depicted in FIG. 11B, the pressure may be removed and the bellows 312a-b may once again return to their uncompressed configurations, rendering the pre-filled multi-liquid medical delivery assembly 300 empty, inert, and/or ready for disposal.

In some embodiments, fewer or more components 310-384 and/or various configurations of the depicted components 310-384 may be included in the pre-filled multi-liquid medical delivery assembly 300 without deviating from the scope of embodiments described herein. In some embodiments, the components 310-384 may be similar in configuration and/or functionality to similarly named and/or numbered components as described herein.

### IV. Pre-filled Multi-Fluid Medical Delivery Methods

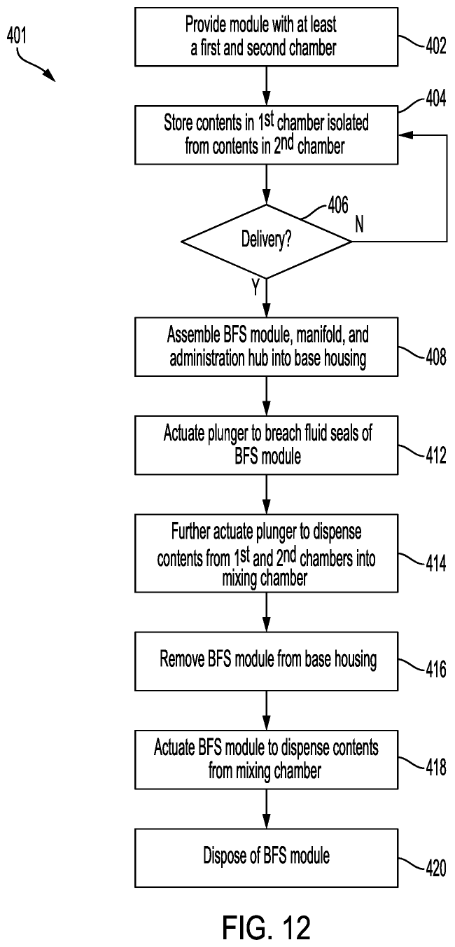

FIG. 12 illustrates an exemplary method 401 for administering a medical treatment using a pre-filled medical treatment system having a parallel configuration. The method 401 can initiate at process block 402, where a BFS module (e.g., a BFS vial) having at least two chambers separately storing respective liquid agents therein is provided. For example, the BFS module can be similar to any of the modules described above with the respect to FIGS. 1A-11B. In some embodiments, the provision of process block 502 can include manufacturing the BFS with the liquid agents sealed therein, for example, using a BFS manufacturing technique.

The method 401 can proceed to process block 404, where the liquid agent contained in a first chamber of the BFS module is stored isolated from the liquid agent contained in a second chamber of the BFS module. In some embodiments, the storing of process block 404 can include providing appropriate environment conditions (e.g., a temperature at or below room temperature (e.g., 20-22° C.)) for the module to maintain viability of the liquid agents contained therein. For example, the BFS module can be maintained in an as-manufactured sealed configuration, with the contents of the first and second chambers separated from each other and the environment until combination of the separate liquid agents is desired (e.g., just prior to administration to a patient), as determined at decision block 406.

If administration of the liquid agents is desired at decision block 406, the method 401 can proceed to process block 408; otherwise, the method 401 returns to process block 404 to maintain the BFS module in a sealed state. At process block 408, the BFS module can be assembled together with a manifold (e.g., manifold 108 or 330), and optionally an administration assembly (e.g., administration hub 104 or 350, and administration member 106 or 356), into a base housing (e.g., base housing 102 or 360). In some embodiments, the administration assembly can be coupled to an outlet longitudinal end of the manifold, and then inserted through a top longitudinal end of the base housing. The BFS module can then be inserted through the top longitudinal end of the base housing and into the inlet longitudinal end of the manifold, such that piercing elements within the manifold are aligned with, but not penetrating, corresponding sealed ports of liquid-agent-containing chambers of the BFS module. Alternatively, in some embodiments, the BFS module can be inserted into the inlet longitudinal end of the manifold prior to the manifold being inserted into the base housing, and before or after coupling of the administration assembly to the manifold. In some embodiments, process block 408 can further include enclosing the BFS module and the manifold within the base housing by inserting a plunger assembly (e.g., the plunger first member 126 and plunger second member 124 of FIGS. 1A-1D, or the end cap 370 and plunger 380 of FIGS. 3A-11B) at the top longitudinal end of the base housing. In such embodiments, the plunger assembly may avoid pressing on the BFS module (e.g., the actuation members and/or the bridge member) to keep the liquid-agent-containing chambers of the BFS module sealed.

The method 401 can proceed to process block 412, where fluid seals of the BFS module are opened by actuation of a plunger assembly. For example, a user can longitudinally displace (e.g., by pressing) a plunger assembly, or component thereof (e.g., the plunger second member 124 or plunger 380), to push the BFS module into contact with the piercing elements of the manifold, such that the sealed ports are pierced, broken, punctured, ruptured, or otherwise breached by the piercing elements, thereby providing fluid communication between the liquid-agent-containing chambers of the BFS module and the mixing chamber of the manifold.

The method 401 can proceed to process block 414, where the contents of the liquid-agent-containing chambers of the BFS module are dispensed into the mixing chamber of the manifold by further actuation of the plunger assembly. For example, a user can further longitudinally displace (e.g., by pressing or screwing) the plunger assembly, or component thereof (e.g., the plunger first member 126 or end cap 370), to impact and compress actuation members of the BFS module, such that the resulting pressure forces contents of the liquid-agent-containing chambers through the breached ports into the manifold. In some embodiments, the medical delivery assembly can be shaken after the contents are forced into the manifold, for example to ensure sufficient emptying of chambers of the BFS module and/or adequate mixing of the fluids in the mixing chamber of the manifold.

The method 401 can proceed to process block 416, where the BFS module and the manifold are removed from the base housing. For example, a user can longitudinally displace (e.g., by retracting and/or unscrewing) the plunger assembly, or component thereof, from the top end of the base housing, exposing the internal volume of the base housing and the BFS module and manifold therein. The removal of the plunger assembly can further allow the actuation members of the BFS module to revert to their respective uncompressed states. The user can then longitudinally displace the BFS module and manifold (and administration assembly coupled thereto) through the top end of the base housing in preparation for administration of the combined liquid agents to a patient.

The method 401 can proceed to process block 418, where the combined liquid agents contained in the mixing chamber of the manifold can be administered to a patient. If the administration assembly includes a protective cap, it can be removed and the administration member inserted into the patient. The user can then re-compress the actuation members of the BFS module, which compression pressurizes the chambers of the BFS module and the mixing chamber of the manifold to cause dispensing of contents thereof via the administration assembly into the patient. In some embodiments, the administration member can comprise a needle, and the dispensing of process block 418 can include inserting an outlet end of the needle into the patient, for example, to effect a subcutaneous, intramuscular, intradermal, and intravenous injection of the combined liquid agents. Alternatively, in some embodiments, the administration member can comprise a nozzle, and the dispensing of process block 418 can include inserting an outlet end of the nozzle into an orifice or cavity of the patient (e.g., oral, nasal, ear canal, etc.) or disposing adjacent to an exposed surface of the patient (e.g., for topical application, eyes, etc.) In some embodiments, the dispensing of combined liquid agents of process block 514 is effective to a deliver a single dose of a therapeutic agent to the patient, for example, having a volume of 1.5 ml or less (e.g., 0.5 ml to 1 ml, inclusive). After administration, the administration assembly can be removed from the patient, for example, while maintaining the actuation members of the BFS module in a compressed state.

The method 401 can proceed to process block 420, where some or all of the components of the pre-filled medical treatment system can be disposed. For example, the BFS module can be constructed for single use and may be incapable of (or at least undesirable for) reuse once the seals are breached and the liquid agents are dispensed therefrom. In some embodiments, at least the manifold of the mixing assembly can also be constructed for single use and may be discarded after the administration. Alternatively or additionally, in some embodiments, the mixing assembly or parts thereof (e.g., base housing 360, end cap 370, and/or plunger 380) can be retained for reuse, with or without cleaning and/or sterilization.

Although some of blocks 402-420 of method 401 have been described as being performed once, in some embodiments, multiple repetitions of a particular process block may be employed before proceeding to the next decision block or process block. In addition, although blocks 402-420 of method 401 have been separately illustrated and described, in some embodiments, process blocks may be combined and performed together (simultaneously or sequentially). Moreover, although FIG. 4 illustrates a particular order for blocks 402-420, embodiments of the disclosed subject matter are not limited thereto. Indeed, in certain embodiments, the blocks may occur in a different order than illustrated or simultaneously with other blocks. For example, in some embodiments, the assembly of process block 408 may occur prior to the storage of process block 404 and the delivery decision of decision block 406, for example, where the medical delivery assembly is provided from a factory in an assembled and sealed state (e.g., configuration 100 of FIG. 1A or initial state of assembly 300 in FIGS. 3C-3D).

### V. Additional Examples of the Disclosed Technology

In view of the above described implementations of the disclosed subject matter, this application discloses the additional examples in the clauses enumerated below. It should be noted that one feature of a clause in isolation, or more than one feature of the clause taken in combination, and, optionally, in combination with one or more features of one or more further clauses are further examples also falling within the disclosure of this application.

Clause 1. A pre-filled multi-fluid medical delivery assembly, comprising:

a blow-fill-seal (BFS) module defining at least two collapsible fluid chambers, each collapsible fluid chamber being in fluid communication with an axially-aligned fluid reservoir at a first end of the reservoirs and each fluid reservoir being sealed at a second end of the reservoirs;

a mixing connector comprising a chamber into which each of the fluid reservoirs may be seated, a piercing element coupled to align with the sealed second ends of the reservoirs in the case that the reservoirs are disposed in the chambers, an internal volume defining a mixing chamber, and a neck comprising threads and defining an outlet port;

a needle hub comprising threads that are cooperatively mated with the threads of the neck of the mixing connector, the needle hub being coupled to a needle disposed through the needle hub;

a cap covering an administration end of the needle; and

an outer cover in which the BFS module, the mixing connector, and the needle hub are disposed, the outer cover comprising a main body portion and a selectively mated end cap.

Clause 2. The assembly of any clause or example herein, in particular clause 1, wherein engagement of the end cap with the main body portion of the outer housing causes the piercing elements to pierce the respective fluid reservoirs, thereby releasing the fluids stored therein into the mixing chamber.

Clause 3. A pre-filled medical delivery assembly comprising:

a blow-fill-seal (BFS) module having first and second longitudinal ends, the BFS module comprising:
first and second chambers, the first chamber having a first liquid agent therein, the second chamber having a second liquid agent therein;
first and second sealed ports proximal to the first longitudinal end of the BFS module, the first sealed port being in fluid communication with the first chamber, the second sealed port being in fluid communication with the second chamber; and
first and second actuation members proximal to the second longitudinal end of the BFS module, the first actuation member being in fluid communication with the first chamber, the second actuation member being in fluid communication with the second chamber; and

means for piercing the sealed ports and combining the first and second liquid agents in a mixing chamber.

Clause 4. The assembly of any clause or example herein, in particular clause 3, wherein the means for piercing the sealed ports and combining the first and second liquid agents further comprises means for administering the combination to a patient.

Clause 5. A pre-filled medical delivery assembly comprising:

(i) a blow-fill-seal (BFS) module having first and second longitudinal ends, the BFS module comprising:
first and second chambers;
first and second ports proximal to the first longitudinal end of the BFS module, the first port being in fluid communication with the first chamber, the second port being in fluid communication with the second chamber; and
first and second actuation members proximal to the second longitudinal end of the BFS module, the first actuation member being in fluid communication with the first chamber, the second actuation member being in fluid communication with the second chamber; and

(ii) a manifold having fifth and sixth longitudinal ends, the manifold defining a second internal volume extending from the fifth longitudinal end to the sixth longitudinal end, a portion of the second internal volume proximal to the fifth longitudinal end being a mixing chamber for first and second liquid agents previously dispensed from the first and second chambers via the first and second ports,

wherein the first longitudinal end of the BFS module is disposed within the sixth longitudinal end of the manifold,

the manifold has first and second longitudinally-extending piercing elements aligned and extending through the first and second sealed ports, respectively.

Clause 6. The assembly of any clause or example herein, in particular clause 5, further comprising:

(iii) an administration assembly comprising:
a hub disposed proximal to the fifth longitudinal end of the manifold; and
at least one administration conduit having an outlet at an end thereof longitudinally spaced from the hub and being in fluid communication with the mixing chamber so as to receive a combination of the first and second liquid agents therefrom.

Clause 7. The assembly of any clause or example herein, in particular clause 6, wherein the at least one administration conduit comprises a needle or cannula.

Clause 8. The assembly of any clause or example herein, in particular clause 6, wherein the outlet of the at least one administration conduit is formed as a nozzle configured to facilitate dispersion of the combination of the first and second liquid agents into a spray, or the outlet of the at least one administration conduit is formed as a nozzle configured to facilitate dispersion of the combination of the first and second liquid agents into one or more droplets.

Clause 9. A pre-filled medical delivery assembly comprising:

(i) a blow-fill-seal (BFS) module having first and second longitudinal ends, the BFS module comprising:
first and second chambers, the first chamber having a first liquid agent therein, the second chamber having a second liquid agent therein;
first and second sealed ports proximal to the first longitudinal end of the BFS module, the first sealed port being in fluid communication with the first chamber, the second sealed port being in fluid communication with the second chamber; and
first and second actuation members proximal to the second longitudinal end of the BFS module, the first actuation member being in fluid communication with the first chamber, the second actuation member being in fluid communication with the second chamber; and

(ii) a mixing assembly constructed to be coupled to the BFS module, the mixing assembly comprising:
a base housing having third and fourth longitudinal ends, the base housing defining a first internal volume extending from the third longitudinal end to the fourth longitudinal end;
a manifold having fifth and sixth longitudinal ends, the manifold defining a second internal volume extending from the fifth longitudinal end to the sixth longitudinal end, the fifth longitudinal end being disposed proximal to the third longitudinal end of the base housing, a portion of the second internal volume proximal to the fifth longitudinal end being sized and shaped to act as a mixing chamber for the first and second liquid agents, the sixth longitudinal end being sized and shaped to receive at least the first longitudinal end of the BFS module therein, the manifold comprising first and second longitudinally-extending piercing elements disposed within the second internal volume to align with the first and second sealed ports, respectively; and
a plunger comprising a laterally-extending first member and a longitudinally-extending second member, the fourth longitudinal end of the base housing being sized and shaped to receive the plunger therein.

Clause 10. The assembly of any clause or example herein, in particular, any one of clauses 3-9, wherein the first liquid agent, the second liquid agent, or both comprise a vaccine, a drug, a medicament, or a component of any of the foregoing.

Clause 11. The assembly of any clause or example herein, in particular, any one of clauses 3-10, wherein a seal of each of the first and second sealed ports comprises a foil, wax, paper, a section of the BFS module, or any combination of the foregoing.

Clause 12. The assembly of any clause or example herein, in particular, any one of clauses 3-11, wherein each of the first and second actuation members comprises a deformable or collapsible chamber.

Clause 13. The assembly of any clause or example herein, in particular, any one of clauses 3-12, wherein one or both of the first and second actuation members are shaped as bellows.

Clause 14. The assembly of any clause or example herein, in particular, any one of clauses 3-13, wherein the first and second actuation members are constructed to be independently actuatable.

Clause 15. The assembly of any clause or example herein, in particular, any one of clauses 5-14, wherein at least a portion of the fifth longitudinal end of the manifold is disposed in or extends through the third longitudinal end of the base housing.

Clause 16. The assembly of any clause or example herein, in particular, any one of clauses 9-15, wherein, with the plunger received in the fourth longitudinal end of the base housing, a bottom surface of the first member of the plunger faces or is in contact with the first and second actuation members, and a portion of the second member of the plunger extends longitudinally between the first and second actuation members.

Clause 17. The assembly of any clause or example herein, in particular clause 16, wherein the BFS module further comprises a bridge member that connects the first and second chambers along facing lateral surface portions, and the portion of the second member, which extends between the first and second actuation members, contacts the bridge member.

Clause 18. The assembly of any clause or example herein, in particular, any one of clauses 5-17, further comprising:

(iii) an administration assembly comprising:
a hub disposed proximal to the fifth longitudinal end of the manifold; and
at least one administration conduit having an outlet at an end thereof longitudinally spaced from the hub and being in fluid communication with the mixing chamber so as to receive a combination of the first and second liquid agents therefrom.

Clause 19. The assembly of any clause or example herein, in particular clause 18, wherein at least part of the administration assembly is formed separate from and constructed to be coupled to the manifold, or at least part of the administration assembly is integrally formed with the manifold.

Clause 20. The assembly of any clause or example herein, in particular, any one of clauses 18-19, wherein the at least one administration conduit comprises a needle or cannula.

Clause 21. The assembly of any clause or example herein, in particular clause 20, wherein the needle has a length in a range of 0.5 mm to 4 mm, inclusive, or in a range of 4 mm to 15 mm, inclusive, or in a range of 15 mm to 30 mm, inclusive.

Clause 22. The assembly of any clause or example herein, in particular, any one of clauses 18-19, wherein the outlet of the at least one administration conduit is formed as a nozzle configured to facilitate dispersion of the combination of the first and second liquid agents into a spray, or the outlet of the at least one administration conduit is formed as a nozzle configured to facilitate dispersion of the combination of the first and second liquid agents into one or more droplets.

Clause 23. The assembly of any clause or example herein, in particular, any one of clauses 9-22, wherein the manifold, the BFS module, and the administration assembly are constructed to be removed as a unit from the base housing after combination of the first and second liquid agents in the mixing chamber.

Clause 24. The assembly of any clause or example herein, in particular, any one of clauses 5-23, wherein:

a first portion of the second internal volume has a frustoconical or tapered shape that narrows along a direction from the sixth longitudinal end toward to the fifth longitudinal end of the manifold in a cross-sectional view;

a second portion of the second internal volume proximal to the fifth longitudinal end has a cylindrical shape having a first diameter;

a third portion of the second internal volume proximal to the sixth longitudinal end has a shape with a maximum cross-sectional dimension greater than the first diameter; or

any combination of the above.

Clause 25. The assembly of any clause or example herein, in particular clause 24, wherein the first and second piercing elements extend longitudinally from an inclined sidewall of the manifold defining the frustoconical or tapered shape of the first portion.

Clause 26. The assembly of any clause or example herein, in particular, any one of clauses 5-25, wherein a volume of the mixing chamber is greater than or equal to a combined volume of the first and second liquid agents.

Clause 27. The assembly of any clause or example herein, in particular, any one of clauses 9-26, wherein the base housing, the plunger, or both are formed of a material having a hardness greater than that of the manifold.

Clause 28. The assembly of any clause or example herein, in particular, any one of clauses 9-27, wherein the base housing, the plunger, or both are formed of polypropylene; and the manifold is formed of polycarbonate.

Clause 29. The assembly of any clause or example herein, in particular, any one of clauses 9-28, wherein the second member of the plunger is movable along a longitudinal direction with respect to the first member of the plunger.

Clause 30. The assembly of any clause or example herein, in particular, any one of clauses 9-29, wherein:

the first member of the plunger comprises a threaded cylindrical portion; and

the fourth longitudinal end of the base housing has a threaded internal surface portion constructed to engage with threads of the cylindrical portion of the first member.

Clause 31. The assembly of any clause or example herein, in particular, any one of clauses 9-30, wherein the second member of the plunger is disposed such that longitudinal displacement of the second member toward the third longitudinal end of the base housing pushes the BFS module toward the manifold, so as to breach the first and second sealed ports using the piercing elements without pressing the first and second actuation members.

Clause 32. The assembly of any clause or example herein, in particular, any one of clauses 9-31, wherein the first member of the plunger is disposed such that longitudinal displacement of the first member toward the third longitudinal end of the base housing presses the first and second actuation members of the BFS module, so as to cause the first and second liquid agents to be dispensed from the first and second chambers into the mixing chamber via the breached ports.

Clause 33. The assembly of any clause or example herein, in particular, any one of clauses 9-32, wherein each of the first and second longitudinally-extending piercing elements is substantially solid, such that the liquid agents from the first and second chambers flow around the respective piercing element extending through the respective breached port.

Clause 34. The assembly of any clause or example herein, in particular, any one of clauses 9-33, wherein at least the manifold and the BFS module are constructed to be removed as a unit from the base housing via the third longitudinal end after combination of the first and second liquid agents in the mixing chamber.

Clause 35. A kit comprising multiple components to be assembled to form the pre-filled medical delivery assembly of any clause or example herein, in particular, any one of clauses 1-34.

Clause 36. A method of administering a medical treatment comprising:

coupling the mixing assembly to the BFS module of a kit to form the pre-filled medical delivery assembly of any clause or example herein, in particular, any one of clause 1-34;

combining the first and second liquid agents; and

administering the combined liquid agents to a patient.

Clause 37. The method of any clause or example herein, in particular clause 36, further comprising, prior to the coupling, filling the first and second liquid agents in the first and second chambers, respectively, using a blow-fill-seal technique.

Clause 38. A method for delivery of liquid agents to a patient, comprising:

(a) providing a blow-fill-seal (BFS) module having first and second longitudinal ends, the BFS module comprising:
first and second chambers, the first chamber having a first liquid agent therein, the second chamber having a second liquid agent therein;
first and second sealed ports proximal to the first longitudinal end of the BFS module, the first sealed port being in fluid communication with the first chamber, the second sealed port being in fluid communication with the second chamber; and
first and second actuation members proximal to the second longitudinal end of the BFS module, the first actuation member being in fluid communication with the first chamber, the second actuation member being in fluid communication with the second chamber;

(b) inserting the first longitudinal end of the BFS module into a sixth longitudinal end of a manifold such that the first and second sealed ports are aligned with first and second longitudinally-extending piercing elements within the manifold, the manifold defining a second internal volume extending from a fifth longitudinal end thereof to the sixth longitudinal end, a portion of the second internal volume proximal to the fifth longitudinal end defining a mixing chamber;

(c) inserting the fifth longitudinal end of the manifold into a third longitudinal end of a base housing, the base housing having the third longitudinal end and a fourth longitudinal end, the base housing defining a first internal volume extending from the third longitudinal end to the fourth longitudinal end;

(d) inserting a plunger into the third longitudinal end of the base housing, the plunger comprising a laterally-extending first member and a longitudinally-extending second member;

(e) after (d), displacing the second member of the plunger longitudinally toward the third longitudinal end of the base housing so as to push the BFS module toward the third longitudinal end of the manifold without compressing the first and second actuation members, such that the first and second sealed ports are breached by the piercing elements;

(f) after (e), displacing the first member of the plunger longitudinally toward the third longitudinal end of the base housing so as to compress the first and second actuation members of the BFS module, such that the first and second liquid agents are dispensed from the first and second chambers into the mixing chamber via the breached ports;

(g) after (f), removing the plunger from the base housing, such that the first and second actuation members of the BFS module revert to respective uncompressed states;

(h) after (g), removing the manifold and the BFS module as a unit from the base housing; and

(i) after (h), re-compressing the first and second actuation members to pressurize the first chamber, the second chamber, and the mixing chamber to cause dispensing of contents thereof.

Clause 39. The method of any clause or example herein, in particular clause 38, further comprising:

prior to (i), coupling a hub of an administration assembly to the fifth longitudinal end of the manifold, the administration assembly further comprising at least one administration conduit having an outlet at an end thereof longitudinally spaced from the hub and being in fluid communication with the mixing chamber so as to receive a combination of the first and second liquid agents therefrom.

Clause 40. The method of any clause or example herein, in particular clause 39, wherein the coupling of the administration assembly to the manifold is prior to (d).

Clause 41. The method of any clause or example herein, in particular, any one of clauses 39-40, wherein:

the at least one administration conduit comprises a needle or cannula; and

the method further comprises, after (h) and prior to (i), inserting an outlet end of the needle or cannula into a patient.

Clause 42. The method of any clause or example herein, in particular clause 41, wherein the needle has a length in a range of 0.5 mm to 4 mm, inclusive, or in a range of 4 mm to 15 mm, inclusive, or in a range of 15 mm to 30 mm, inclusive.

Clause 43. The method of any clause or example herein, in particular, any one of clauses 39-40, wherein:

the outlet of the at least one administration conduit is formed as a nozzle configured to facilitate dispersion of the combination of the first and second liquid agents into a spray, or the outlet of the at least one administration conduit is formed as a nozzle configured to facilitate dispersion of the combination of the first and second liquid agents into one or more droplets; and

the method further comprises, after (h) and prior to (i), inserting an outlet end of the nozzle into an orifice of a patient.

Clause 44. The method of any clause or example herein, in particular, any one of clauses 38-43, wherein a seal of each of the first and second sealed ports comprises a foil, wax, paper, a section of the BFS module, or any combination of the foregoing.

Clause 45. The method of any clause or example herein, in particular, any one of clauses 38-44, wherein each of the first and second actuation members comprises a deformable or collapsible chamber.

Clause 46. The method of any clause or example herein, in particular, any one of clauses 38-45, wherein one or both of the first and second actuation members are shaped as bellows.

Clause 47. The method of any clause or example herein, in particular, any one of clauses 38-46, wherein the first and second actuation members are independently actuatable.

Clause 48. The method of any clause or example herein, in particular, any one of clauses 38-47, wherein, during (e), a bottom surface of the first member of the plunger faces or is in contact with the first and second actuation members, while a portion of the second member of the plunger extends longitudinally between the first and second actuation members.

Clause 49. The method of any clause or example herein, in particular, any one of clauses 38-48, wherein:

the BFS module further comprises a bridge member that connects the first and second chambers along facing lateral surface portions; and

during (e), the portion of the second member that extends between the first and second actuation members pushes on the bridge member to move the BFS module toward the third longitudinal end of the manifold.

Clause 50. The method of any clause or example herein, in particular, any one of clauses 38-49, wherein:

the first member of the plunger comprises a threaded cylindrical portion;

the fourth longitudinal end of the base housing has a threaded internal surface portion constructed to engage with threads of the cylindrical portion of the first member;

the displacing of (f) comprises screwing the threaded cylindrical portion into the fourth longitudinal end of the base housing; and

the removing of (g) comprises unscrewing the threaded cylindrical portion from the fourth longitudinal end of the base housing.

Clause 51. The method of any clause or example herein, in particular, any one of clauses 36-50, wherein the first liquid agent, the second liquid agent, or both comprise a vaccine, a drug, a medicament, or a component of any of the foregoing.

Clause 52. The method of any clause or example herein, in particular, any one of clauses 36-51, wherein the administering is via subcutaneous, intramuscular, intradermal, and intravenous injection of the combined liquid agents.

Clause 53. The method of any clause or example herein, in particular, any one of clauses 36-52, wherein:

a combined volume of all of liquid-agent-containing chambers of the BFS module is in a range of 0.50 ml to 0.75 ml, inclusive;

a combined volume of all liquid agents in the BFS module is in a range of 0.50 ml to 0.75 ml, inclusive; or both of the above.

Clause 54. The method of any clause or example herein, in particular, any one of Clauses 36-53, wherein the administering delivers the combined liquid agents as a single dose of a therapeutic agent to the patient.

### VI. Rules of Interpretation

Any of the features illustrated or described with respect to FIGS. 1A-12 and Clauses 1-54 can be combined with any other features illustrated or described with respect to FIGS. 1A-12 and Clauses 1-54 to provide systems, assemblies, kits, devices, methods, and embodiments not otherwise illustrated or specifically described herein. For example, the nozzle for the administration assembly can be applied to any other manifold or administration assembly, described herein or otherwise, for example, the manifold 330 of FIGS. 3A-11B, or the administration hub 350 of FIGS. 3A-11B. Other combinations and variations are also possible according to one or more contemplated embodiments. All features described herein are independent of one another and, except where structurally impossible, can be used in combination with any other feature described herein.

Any or all of the components disclosed herein can be formed of one or more plastics. In some embodiments, some components (e.g., the BFS vials) can be formed of a relatively soft polymer (e.g., having a Shore/Durometer “D” hardness of between 60 and 70), such as polyethylene, polypropylene, or any other polymer adaptable for use in a BFS manufacturing process. In some embodiments, some components (e.g., the connection assemblies, the administration assemblies, and/or needle caps or covers) can be formed, at least in part, of a relatively hard polymer (e.g., having a hardness greater than 80 on the Rockwell “R” scale), such as, but not limited to, polybenzimidazole, acrylonitrile butadiene styrene (ABS), polystyrene, polyvinyl chloride, or the like. Other materials are also possible according to one or more contemplated embodiments.

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments of the invention nor a listing of features of the invention that must be present in all embodiments.

Neither the Title (set forth at the beginning of the first page of this patent application) nor the Abstract (set forth at the end of this patent application) is to be taken as limiting in any way as the scope of the disclosed invention(s).

While the term “vial” is utilized herein for convenience and ease of illustration, objects represented and/or described as “vials” may comprise various forms, configurations, and/or quantities of components. A BFS vial may comprise one or more BFS products that are formed and/or manufactured together or separately, for example, and/or may comprise one or more BFS chambers, bottles, containers, and/or other fluid-retaining objects. The term “vial” does not convey any designation of shape or size. In some embodiments, a BFS component may comprise one or more vials. According to some embodiments a BFS component and/or a BFS vial may comprise one or more fluid chambers. In some embodiments, a plurality of BFS components, vials, and/or chambers may be manufactured simultaneously from a single BFS mold. Each respective vial and/or chamber may be formed, for example, by different portions of a single BFS mold (e.g., two cooperative halves thereof). In some embodiments, BFS components, vials, and/or chambers may be joined and/or coupled during manufacturing (e.g., via unformed and/or fused connecting parison) and/or after manufacturing/filling.

The term “product” means any machine, manufacture and/or composition of matter as contemplated by 35 U.S.C. § 101, unless expressly specified otherwise.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

A reference to “another embodiment” in describing an embodiment does not imply that the referenced embodiment is mutually exclusive with another embodiment (e.g., an embodiment described before the referenced embodiment), unless expressly specified otherwise.

The terms “a”, “an” and “the” mean “one or more”, unless expressly specified otherwise.

The term “plurality” means “two or more”, unless expressly specified otherwise.

The term “herein” means “in the present application, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device or article is described herein, more than one device or article (whether or not they cooperate) may alternatively be used in place of the single device or article that is described. Accordingly, the functionality that is described as being possessed by a device may alternatively be possessed by more than one device or article (whether or not they cooperate).

Similarly, where more than one device or article is described herein (whether or not they cooperate), a single device or article may alternatively be used in place of the more than one device or article that is described. For example, a plurality of computer-based devices may be substituted with a single computer-based device. Accordingly, the various functionality that is described as being possessed by more than one device or article may alternatively be possessed by a single device or article.

The functionality and/or the features of a single device that is described may be alternatively embodied by one or more other devices which are described but are not explicitly described as having such functionality and/or features. Thus, other embodiments need not include the described device itself, but rather can include the one or more other devices which would, in those other embodiments, have such functionality/features.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

Headings of sections provided in this patent application and the title of this patent application are for convenience only, and are not to be taken as limiting the disclosure in any way.

“Determining” something can be performed in a variety of manners and therefore the term “determining” (and like terms) includes calculating, computing, deriving, looking up (e.g., in a table, database or data structure), ascertaining and the like

The terms “including”, “comprising” and variations thereof mean “including but not limited to”, unless expressly specified otherwise. As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including ten percent (10%) of that value are intended, unless explicitly stated otherwise.

Directions and other relative references may be used to facilitate discussion of the drawings and principles herein, but are not intended to be limiting. For example, certain terms may be used such as “inner,” “outer,”, “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A pre-filled medical delivery assembly comprising:
(i) a blow-fill-seal (BFS) module having first and second longitudinal ends, the BFS module comprising:
first and second chambers, the first chamber having a first liquid agent therein, the second chamber having a second liquid agent therein;
first and second sealed ports proximal to the first longitudinal end of the BFS module, the first sealed port being in fluid communication with the first chamber, the second sealed port being in fluid communication with the second chamber; and
first and second actuation members proximal to the second longitudinal end of the BFS module, the first actuation member being in fluid communication with the first chamber, the second actuation member being in fluid communication with the second chamber; and
(ii) a mixing assembly constructed to be coupled to the BFS module, the mixing assembly comprising:
a base housing having third and fourth longitudinal ends, the base housing defining a first internal volume extending from the third longitudinal end to the fourth longitudinal end;
a manifold having fifth and sixth longitudinal ends, the manifold defining a second internal volume extending from the fifth longitudinal end to the sixth longitudinal end, the fifth longitudinal end being disposed proximal to the third longitudinal end of the base housing, a portion of the second internal volume proximal to the fifth longitudinal end being sized and shaped to act as a mixing chamber for the first and second liquid agents, the sixth longitudinal end being sized and shaped to receive at least the first longitudinal end of the BFS module therein, the manifold comprising first and second longitudinally-extending piercing elements disposed within the second internal volume to align with the first and second sealed ports, respectively; and
a plunger comprising a laterally-extending first member and a longitudinally-extending second member, the fourth longitudinal end of the base housing being sized and shaped to receive the plunger therein.

2. The pre-filled medical delivery assembly of claim 1, wherein the first liquid agent, the second liquid agent, or both comprise a vaccine, a drug, a medicament, or a component of any of the foregoing.

3. The pre-filled medical delivery assembly of claim 1, wherein a seal of each of the first and second sealed ports comprises a foil, wax, paper, a section of the BFS module, or any combination of the foregoing.

4. The pre-filled medical delivery assembly of claim 1, wherein each of the first and second actuation members comprises a deformable or collapsible chamber.

5. The pre-filled medical delivery assembly of claim 4, wherein one or both of the first and second actuation members are shaped as bellows.

6. The pre-filled medical delivery assembly of claim 4, wherein the first and second actuation members are constructed to be independently actuatable.

7. The pre-filled medical delivery assembly of claim 1, wherein at least a portion of the fifth longitudinal end of the manifold is disposed in or extends through the third longitudinal end of the base housing.

8. The pre-filled medical delivery assembly of claim 1, wherein, with the plunger received in the fourth longitudinal end of the base housing, a bottom surface of the first member of the plunger faces or is in contact with the first and second actuation members, and a portion of the second member of the plunger extends longitudinally between the first and second actuation members.

9. The pre-filled medical delivery assembly of claim 8, wherein the BFS module further comprises a bridge member that connects the first and second chambers along facing lateral surface portions, and the portion of the second member, which extends between the first and second actuation members, contacts the bridge member.

10. The pre-filled medical delivery assembly of claim 1, further comprising:
(iii) an administration assembly comprising:
a hub disposed proximal to the fifth longitudinal end of the manifold; and
at least one administration conduit having an outlet at an end thereof longitudinally spaced from the hub and being in fluid communication with the mixing chamber so as to receive a combination of the first and second liquid agents therefrom.

11. The pre-filled medical delivery assembly of claim 10, wherein at least part of the administration assembly is formed separate from and constructed to be coupled to the manifold, or at least part of the administration assembly is integrally formed with the manifold.

12. The pre-filled medical delivery assembly of claim 10, wherein the at least one administration conduit comprises a needle or cannula.

13. The pre-filled medical delivery assembly of claim 12, wherein the needle has a length in a range of 0.5 mm to 4 mm, inclusive, or in a range of 4 mm to 15 mm, inclusive, or in a range of 15 mm to 30 mm, inclusive.

14. The pre-filled medical delivery assembly of claim 10, wherein the outlet of the at least one administration conduit is formed as a nozzle configured to facilitate dispersion of the combination of the first and second liquid agents into a spray, or the outlet of the at least one administration conduit is formed as a nozzle configured to facilitate dispersion of the combination of the first and second liquid agents into one or more droplets.

15. The pre-filled medical delivery assembly of claim 10, wherein the manifold, the BFS module, and the administration assembly are constructed to be removed as a unit from the base housing after combination of the first and second liquid agents in the mixing chamber.

16. The pre-filled medical delivery assembly of claim 1, wherein:
a first portion of the second internal volume has a frustoconical or tapered shape that narrows along a direction from the sixth longitudinal end toward to the fifth longitudinal end of the manifold in a cross-sectional view;
a second portion of the second internal volume proximal to the fifth longitudinal end has a cylindrical shape having a first diameter;
a third portion of the second internal volume proximal to the sixth longitudinal end has a shape with a maximum cross-sectional dimension greater than the first diameter; or
any combination of the above.

17. The pre-filled medical delivery assembly of claim 16, wherein the first and second piercing elements extend longitudinally from an inclined sidewall of the manifold defining the frustoconical or tapered shape of the first portion.

18. The pre-filled medical delivery assembly of claim 1, wherein a volume of the mixing chamber is greater than or equal to a combined volume of the first and second liquid agents.

19. The pre-filled medical delivery assembly of claim 1, wherein the base housing, the plunger, or both are formed of a material having a hardness greater than that of the manifold.

20. The pre-filled medical delivery assembly of claim 19, wherein:
the base housing, the plunger, or both are formed of polypropylene; and
the manifold is formed of polycarbonate.

21. The pre-filled medical delivery assembly of claim 1, wherein the second member of the plunger is movable along a longitudinal direction with respect to the first member of the plunger.

22. The pre-filled medical delivery assembly of claim 1, wherein:
the first member of the plunger comprises a threaded cylindrical portion; and
the fourth longitudinal end of the base housing has a threaded internal surface portion constructed to engage with threads of the cylindrical portion of the first member.

23. The pre-filled medical delivery assembly of claim 1, wherein the second member of the plunger is disposed such that longitudinal displacement of the second member toward the third longitudinal end of the base housing pushes the BFS module toward the manifold, so as to breach the first and second sealed ports using the piercing elements without pressing the first and second actuation members.

24. The pre-filled medical delivery assembly of claim 23, wherein the first member of the plunger is disposed such that longitudinal displacement of the first member toward the third longitudinal end of the base housing presses the first and second actuation members of the BFS module, so as to cause the first and second liquid agents to be dispensed from the first and second chambers into the mixing chamber via the breached ports.

25. The pre-filled medical delivery assembly of claim 24, wherein each of the first and second longitudinally-extending piercing elements is substantially solid, such that the liquid agents from the first and second chambers flow around the respective piercing element extending through the respective breached port.

26. The pre-filled medical delivery assembly of claim 1, wherein at least the manifold and the BFS module are constructed to be removed as a unit from the base housing via the third longitudinal end after combination of the first and second liquid agents in the mixing chamber.

27. A method for delivery of liquid agents to a patient, comprising:
(a) providing a blow-fill-seal (BFS) module having first and second longitudinal ends, the BFS module comprising:
first and second chambers, the first chamber having a first liquid agent therein, the second chamber having a second liquid agent therein;
first and second sealed ports proximal to the first longitudinal end of the BFS module, the first sealed port being in fluid communication with the first chamber, the second sealed port being in fluid communication with the second chamber; and
first and second actuation members proximal to the second longitudinal end of the BFS module, the first actuation member being in fluid communication with the first chamber, the second actuation member being in fluid communication with the second chamber;
(b) inserting the first longitudinal end of the BFS module into a sixth longitudinal end of a manifold such that the first and second sealed ports are aligned with first and second longitudinally-extending piercing elements within the manifold, the manifold defining a second internal volume extending from a fifth longitudinal end thereof to the sixth longitudinal end, a portion of the second internal volume proximal to the fifth longitudinal end defining a mixing chamber;
(c) inserting the fifth longitudinal end of the manifold into a third longitudinal end of a base housing, the base housing having the third longitudinal end and a fourth longitudinal end, the base housing defining a first internal volume extending from the third longitudinal end to the fourth longitudinal end;
(d) inserting a plunger into the third longitudinal end of the base housing, the plunger comprising a laterally-extending first member and a longitudinally-extending second member;
(e) after (d), displacing the second member of the plunger longitudinally toward the third longitudinal end of the base housing so as to push the BFS module toward the third longitudinal end of the manifold without compressing the first and second actuation members, such that the first and second sealed ports are breached by the piercing elements;
(f) after (e), displacing the first member of the plunger longitudinally toward the third longitudinal end of the base housing so as to compress the first and second actuation members of the BFS module, such that the first and second liquid agents are dispensed from the first and second chambers into the mixing chamber via the breached ports;
(g) after (f), removing the plunger from the base housing, such that the first and second actuation members of the BFS module revert to respective uncompressed states;
(h) after (g), removing the manifold and the BFS module as a unit from the base housing; and
(i) after (h), re-compressing the first and second actuation members to pressurize the first chamber, the second chamber, and the mixing chamber to cause dispensing of contents thereof.

28. The method of claim 27, further comprising:
prior to (i), coupling a hub of an administration assembly to the fifth longitudinal end of the manifold, the administration assembly further comprising at least one administration conduit having an outlet at an end thereof longitudinally spaced from the hub and being in fluid communication with the mixing chamber so as to receive a combination of the first and second liquid agents therefrom.

29. The method of claim 28, wherein the coupling of the administration assembly to the manifold is prior to (d).

30. The method of claim 28, wherein:
the at least one administration conduit comprises a needle or cannula; and
the method further comprises, after (h) and prior to (i), inserting an outlet end of the needle or cannula into a patient.

31. The method of claim 30, wherein the needle has a length in a range of 0.5 mm to 4 mm, inclusive, or in a range of 4 mm to 15 mm, inclusive, or in a range of 15 mm to 30 mm, inclusive.

32. The method of claim 28, wherein:
the outlet of the at least one administration conduit is formed as a nozzle configured to facilitate dispersion of the combination of the first and second liquid agents into a spray, or the outlet of the at least one administration conduit is formed as a nozzle configured to facilitate dispersion of the combination of the first and second liquid agents into one or more droplets; and
the method further comprises, after (h) and prior to (i), inserting an outlet end of the nozzle into an orifice of a patient.

33. The method of claim 27, wherein the first liquid agent, the second liquid agent, or both comprise a vaccine, a drug, a medicament, or a component of any of the foregoing.

34. The method of claim 27, wherein a seal of each of the first and second sealed ports comprises a foil, wax, paper, a section of the BFS module, or any combination of the foregoing.

35. The method of claim 27, wherein each of the first and second actuation members comprises a deformable or collapsible chamber.

36. The method of claim 35, wherein one or both of the first and second actuation members are shaped as bellows.

37. The method of claim 35, wherein the first and second actuation members are independently actuatable.

38. The method of claim 27, wherein, during (e), a bottom surface of the first member of the plunger faces or is in contact with the first and second actuation members, while a portion of the second member of the plunger extends longitudinally between the first and second actuation members.

39. The method of claim 38, wherein:
the BFS module further comprises a bridge member that connects the first and second chambers along facing lateral surface portions; and
during (e), the portion of the second member that extends between the first and second actuation members pushes on the bridge member to move the BFS module toward the third longitudinal end of the manifold.

40. The method of claim 27 wherein:
the first member of the plunger comprises a threaded cylindrical portion;
the fourth longitudinal end of the base housing has a threaded internal surface portion constructed to engage with threads of the cylindrical portion of the first member;
the displacing of (f) comprises screwing the threaded cylindrical portion into the fourth longitudinal end of the base housing; and
the removing of (g) comprises unscrewing the threaded cylindrical portion from the fourth longitudinal end of the base housing.
